(19) United States
(12) Patent Application Publication
(10) Pub. No.: US 2017/0241931 A1
(43) Pub. Date: Aug. 24, 2017

(54) SENSING SYSTEM

(71) Applicant: Sharp Kabushiki Kaisha, Osaka (JP)

(72) Inventors: TAZUKO KITAZAWA, Osaka (JP); NOBORU IWATA, Osaka (JP); TAKANOBU SATO, Osaka (JP)

(73) Assignee: Sharp Kabushiki Kaisha, Osaka (JP)

(21) Appl. No.: 15/515,090

(22) PCT Filed: Aug. 31, 2015
(86) PCT No.: PCT/JP2015/074737

§ 371 Date: Mar. 28, 2017

(30) Foreign Application Priority Data
Sep. 30, 2014 (JP) 2014-202217

(51) Int. Cl. G01N 27/12; G01N 33/497; G01N 33/00

(52) U.S. Cl. CPC G01N 27/12; G01N 33/0031; G01N 33/4972; G01N 33/004; G01N 33/0047

(57) ABSTRACT

Provided is a sensing system that has a long life span. The sensing system includes a first detector (1) and a second detector (2) that detect at least one type of detection target which is included in a first concept which is common to both of detection targets, and which is included in a subordinate concept of the first concept; and a controller (3) that controls, according to a detected value from any one of the first detector (1) and the second detector (2), start, stop, or a detection condition of detection operations of the other detector.

### TECHNICAL FIELD

The invention relates to a sensing system.

### BACKGROUND ART

Recently, various sensing systems that detect detection targets have been developed. Among these, a technique in order to realize a sensing system having a long life span has been developed.

For example, in PTL 1, a sensitive membrane array-type gas detector having a purpose of obtaining a long life span, long stability, and high detection accuracy is disclosed.

Specifically, a plurality of gas detection elements are provided, and one of the gas detection elements is caused to be in an operation state, and all the others are caused to be in a non-operation state. Also, a function of the gas detection element is the operation state is always checked, and if there is a disorder, the gas detection element is substituted with one of the other gas detection elements. Thereafter, in the same manner, ever time when there is a disorder in the gas detection element in the operation state, the gas detection element is substituted with remaining gas detection elements.

### CITATION LIST

### Patent literature

PTL 1: Japanese Unexamined Patent Application Publication No. 11-160267 (Laid-Open on Jun. 18, 1999)

### Non Patent literature

NPL 1: Study on the Low-cost Process of High Performance Gas Sensing Materials by the Sol-Gel Method, by Masashi SHOYAMA and Noritsugu HASHIMOTO, Research Report of 2002 in Industrial Research Division, Science and Technology Promotion Center, Mie Prefecture No. 27-8 (2003)

NPL 2: Study on the improvement of the gas selectivity of thin-film gas sensors, Kazuhiro HARA, Hidekazu IMAI, Superconductivity Technology Center/High Tech Research Center Research Report (2002)

### SUMMARY OF INVENTION

### Technical Problem

Accordingly, the related art described above has a configuration in which a plurality of gas detection elements for substitution are provided in advance and has a configuration of detecting end of a life span of the gas detection element in use and substituting the detection element to a new gas detection element.

Accordingly, the related art described above has a problem that a plurality of gas detection elements for substitution have to be provided.

The invention has been conceived in view of the problem described above, and the purpose thereof is to provide a sensing system that does not require a plurality of gas detection elements for substitution and that has a long life span.

### Solution to Problem

In order to solve the problem described above, according to one aspect of the invention, there is provided a sensing system including a first detector that detects a first detection target; a second detector that detects a second detection target; and a controller that controls start or stop of detection operations of the first detector and the second detector, in which the first detection target and the second detection target are detection targets included in a first concept and include at least one type of detection target which is common to both of the first detection target and the second detection target and is included in a subordinate concept or the first concept, and, according to a detected value from any one detector of the first detector and the second detector, the controller controls start, stop, or a detection condition of the detection operation of the other detector.

### Advantageous Effects of Invention

According to an aspect of the invention, an effect in which a sensing system has a long life span is exhibited.

### BRIEF DESCRIPTION OF DRAWINGS

FIG. 1 is a diagram illustrating an overview of a sensing system according to the invention.

FIGS. 2(a), 2(b), and 2(c) are diagrams illustrating an example of an external appearance of an alcohol detecting system according to Embodiment 1 of the invention.

FIG. 3 is a functional block diagram schematically illustrating a configuration of the alcohol detecting system according to Embodiment 1 of the invention.

FIGS. 4(a) and 4(b) are examples of a graph illustrating detected values with respect to humidity in a hygrometer and a semiconductor gas sensor according to Embodiment 1 of the invention.

FIGS. 5(a), 5(b) and 5(c) are flow charts illustrating flows of data processes performed by a controller according to Embodiment 1 of the invention.

FIG. 6 is a diagram illustrating an example of an external appearance of an air quality monitoring system according to Embodiment 2 of the invention.

FIG. 7 is a functional block diagram schematically illustrating a configuration of the air quality monitoring system according to Embodiment 2 of the invention.

FIGS. 8(a), 8(b) and 8(c) are flow charts illustrating flows of data processes performed by a controller according to Embodiment 2 of the invention.

FIG. 9 is a graph illustrating sensitivity dependency to an operation temperature (detected temperature) of a gas sensor using a ZnO—SO2 composite thin film.

FIGS. 10(a) and 10(b) are graphs illustrating gas selectivity of a semiconductor gas sensor depending on a combination with Pt or Pd catalyst.

FIG. 11 is a diagram illustrating an example of an external appearance of a gas sensing system according to Embodiment 3 of the invention.

FIG. 12 is a functional block diagram schematically illustrating a configuration of the gas sensing system according to Embodiment 3 of the invention.

FIGS. 13(a) and 13(b) are flow charts illustrating flows of data processes performed by a controller according to Embodiment 3 of the invention.

FIGS. 14(a) and 14(b) are diagrams illustrating an example of an eternal appearance of a light sensing system according to Embodiment 4 of the invention.

FIG. 15 is a functional block diagram schematically illustrating a configuration of the light sensing system according to Embodiment 4 of the invention.

FIGS. 16(a), 16(b) and 16(c) are flow charts illustrating flows of data processes performed by a controller according to Embodiment 4 of the invention.

FIG. 17 is a functional block diagram schematically illustrating a configuration of a light sensing system according to Embodiment 5 of the invention.

FIG. 18 is a flow chart illustrating a flow of data processes performed by a controller according to Embodiment 5 of the invention.

### DESCRIPTION OF EMBODIMENTS

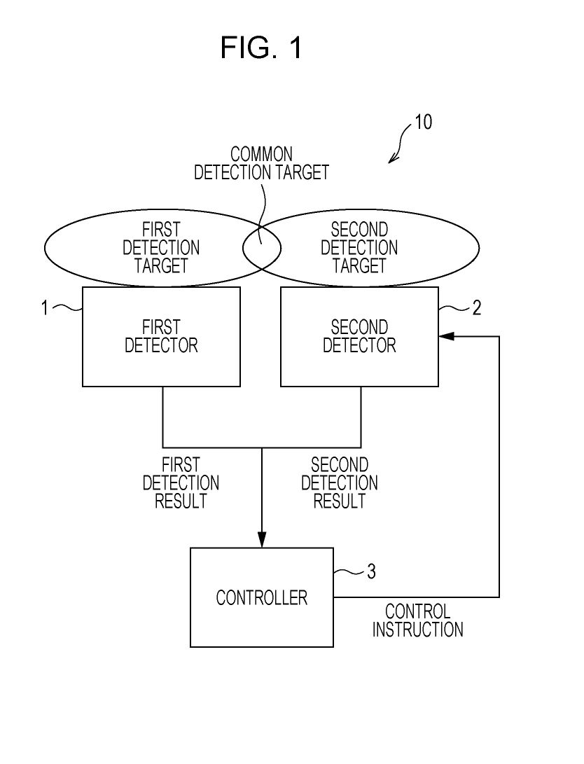

An overview of a sensing system according to the invention is described as follows with reference to FIG. 1. FIG. 1 is a diagram illustrating an overview of a sensing system 10 according to the invention.

(Main Configurations of Sensing System 10)

The sensing system 10 according to the invention includes a first detector 1, a second detector 2, and a controller (control means) 3.

(Detectors 1 and 2)

As the first detector 1 and the second detector 2, a photodetector, a microphone, a piezoelectric element, an ammeter, a voltmeter, a Tesla meter, a thermometer, an ion counter, a Geiger counter, a particle counter, a semiconductor gas sensor, an optical sensor, and a surface plasmon resonance (SPR) sensor can be used.

Detection principles of the first detector 1 and detection principles of the second detector 2 may be identical to or different from each other.

In this specification, parameters that the respective detectors directly detect are set as physical parameters (detection targets), and substances that changes the physical parameters are set as measurement targets, and a purpose of detecting the detection target is set as a detection purpose.

For example, a physical parameter is set as light intensity. The respective detectors detect changes of the inspection light that are generated by causing the inspection light applied to the measurement targets to penetrate the measurement target. The respective detectors detects light (fluorescent light and the like) generated by irradiating the measurement targets with inspection light (excitation light).

The detection purpose is to obtain information relating to a state of the detection targets by analyzing the physical parameters and calculating transmittance and wavelength shifts.

The physical parameters (detection targets) are, for example, electromagnetic wave intensity, sounds, forces, currents, voltages, magnetism, temperatures, and distances.

The detection purpose is to obtain information relating to states of the detection targets or bodies that include or generate the detection targets.

A first detection target and a second detection target are detection targets that are included in the first concept and include at least one type of detection target which is common to both of the first detection target and the second detection target and included in a subordinate concept of the first concept.

At least one of the first detection target or the second detection target includes a detection target other than the detection targets.

(Controller 3)

According to detected values from any one detector of the first detector or the second detector, the controller 3 controls detection conditions of starting, stopping (ending), or detecting the detection operation of the other detector.

Specifically, the controller 3 illustrated in FIG. 1 determines the detected value from one detector and controls a detection start, a detection stop, refreshment, and calibration of the other detector. Otherwise, the controller 3 determines a detected value from one detector and sets conditions of detection start, detection stop, refresh, and calibration of the other detector.

The controller 3 may be realized by a computer. In this case, the controller 3 may be a control program of the sensing system 10 that realizes the sensing system 10 with the computer by operating the computer as respective sections included in the sensing system 10. The controller 3 may be formed only with an electronic circuit.

The sensing system 10 may include a storage section or a display section.

The storage section stores, for example, a detected value from the first detector 1 and/or a detected value from the second detector 2. The storage section may store contents desired by a user who uses the sensing system 10.

The display section displays measured values corresponding to the detected value from the first detector 1 and/or the detected value from the second detector 2. The display section may display the contents desired by the user who uses the sensing system 10.

(Operation of Sensing System)

Subsequently, an operation of the sensing system 10 according to the invention is described. Examples of the operation of the sensing system 10 mainly include three operations below.

In the first operation, only one detector is operated, if the detected value from the corresponding detector is a prescribed value or greater or a prescribed value or less, or a change amount of the detected value from the detector is a prescribed value or greater or a prescribed value or less, the controller 3 causes the operation of the other detector to start.

In the second operation, the first detector 1 and the second detector 2 operate, detection results of one detector is a certain value or greater or a certain value or less or is changed to a certain value or less, the controller 3 stops (ends) the operation of the other detector.

In the third operation, at least one detector operates, and according to the detected value from the corresponding detector, the controller 3 sets the detection condition of the other detector. Refreshment or calibration is also included in this detection condition.

Hereinafter, some configurations of the operation of the sensing system 10 according to the invention are specifically described with reference to embodiments.

### Embodiment 1

The embodiment of the invention is described as follows based on FIGS. 1 to 5. For convenience of explanation, members having the same functions as members described in the embodiment are denoted by the same reference numerals, and the descriptions thereof are omitted.

(External Appearance of Alcohol Detecting System 10a)

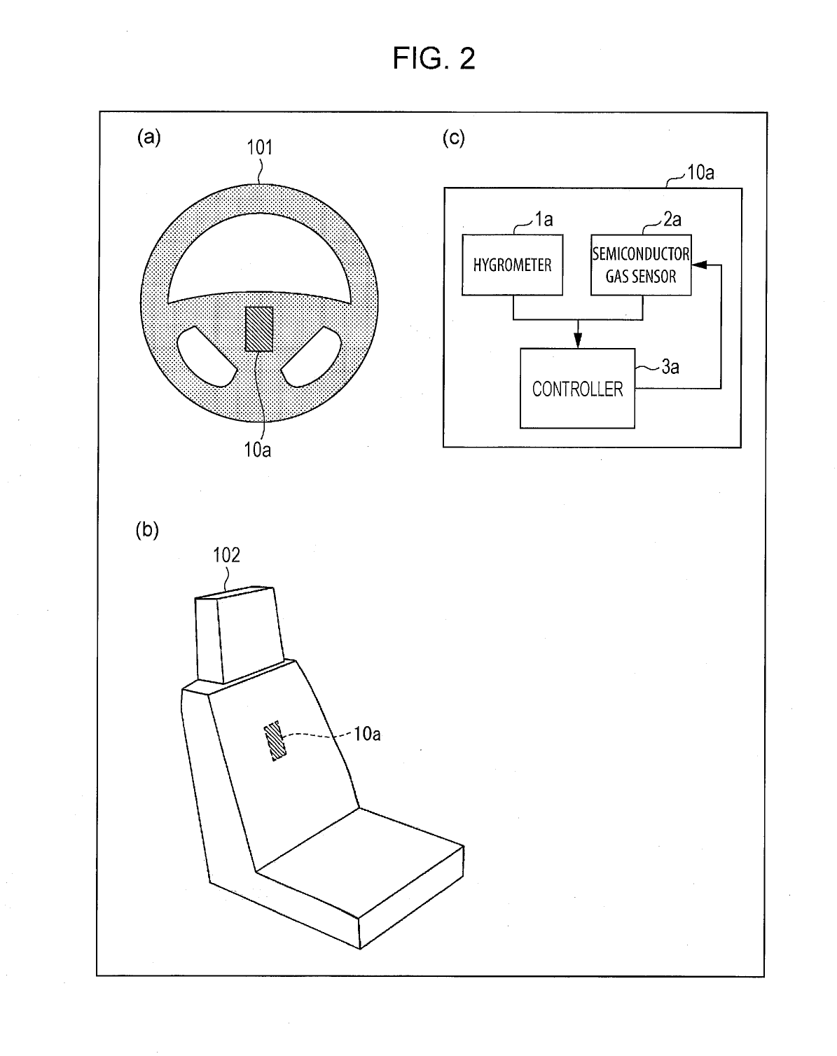

An example of an external appearance of the sensing system according to an embodiment is described by using FIGS. 2(a) to 2(c). The sensing system according to an embodiment is an alcohol detecting system 10a (sensing system). For example, the alcohol detecting system 10a is used for checking a drinking amount or a degree of drunkenness of a human. According to the embodiment, an example in which the alcohol detecting system 10a is used for checking alcohol of a driver in a vehicle is described. The detection purpose according to the embodiment is to obtain information on an ethanol concentration contained in exhaled breath of a human.

For example, FIG. 2(a) illustrates an example in which the alcohol detecting system 10a is included a handle 101 of an automobile. FIG. 2(b) illustrates an example in which the alcohol detecting system 10a is included in a seat 102 of an automobile. FIG. 2(c) is a block diagram schematically illustrating the alcohol detecting system 10a.

As illustrated in FIG. 2(c), the alcohol detecting system 10a detects water vapor included in sweat or exhaled breath of a human who gets in the automobile by a hygrometer 1a (first detector) and starts the detection of a semiconductor gas sensor (second detector) 2a that detects ethanol according to the detection.

(Main Configurations of Alcohol Detecting System 10a)

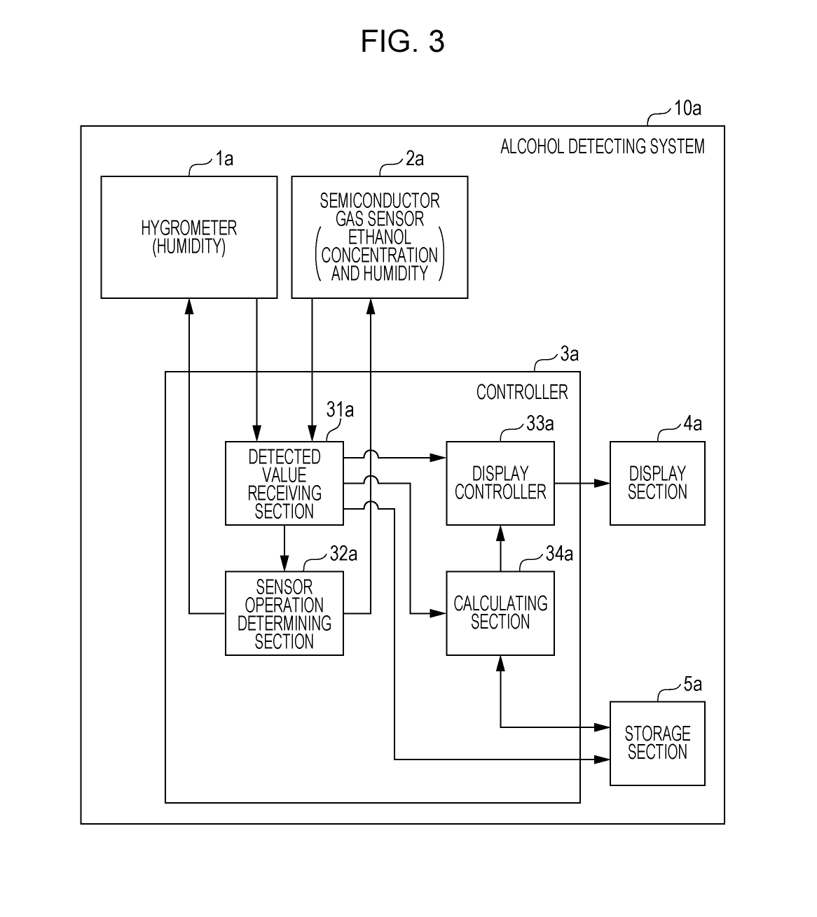

Subsequently, with reference to FIG. 3, main configurations of the alcohol detecting system 10a according to the embodiment are described. FIG. 3 is a functional block diagram schematically illustrating a configuration of the alcohol detecting system 10a. As illustrated in FIG. 3, the alcohol detecting system 10a includes the hygrometer 1a, the semiconductor gas sensor 2a, a controller 3a, a display section 4a, and a storage section 5a.

(Hygrometer 1a)

The hygrometer 1a is a digital hygrometer using changes of electric resistance by absorbing moisture. The hygrometer 1a detects humidity as the detection target. Here, the humidity indicates a water vapor concentration. The hygrometer 1a sends the detected value to the controller 3a.

(Semiconductor Gas Sensor 2a)

The semiconductor gas sensor 2a is a semiconductor gas sensor (semiconductor-type gas sensor and semiconductor film-type gas sensor). The semiconductor gas sensor 2a detects an ethanol concentration and humidity as the detection targets. The semiconductor gas sensor 2a sends the detected values to the controller 3a.

Here, a semiconductor gas sensor, a contact combustion-type gas sensor, an optical gas sensor, and the like detect changes of physical parameters of a reaction film when the reaction film such as an oxide semiconductor reacts with gas. Generally, the physical parameter of the reaction film changes by humidity, and thus the physical parameter is greatly influenced by water vapor. Accordingly, the detection targets of the semiconductor gas sensor 2a include water vapor.

The detection target of the hygrometer 1a and the detection target of the semiconductor gas sensor 2a are detection targets included in the first concept called gas. A common detection target of hygrometer 1a and the semiconductor gas sensor 2a is humidity included in the subordinate concept of the first concept, that is, the water vapor concentration.

(Controller 3a)

The controller 3a includes a detected value receiving section 31a, a sensor operation determining section (control means) 32a, a display controller 33a, and a calculating section (calculating means) 34a.

The detected value receiving section 31a receives detected values from the hygrometer 1a and the semiconductor gas sensor 2a and sends the detected values to the sensor operation determining section 32a, the display controller 33a, the calculating section 34a, and the storage section 5a.

The sensor operation determining section 32a determines detected values from the hygrometer 1a and/or the semiconductor gas sensor 2a and controls operations of the hygrometer 1a or the semiconductor gas sensor 2a.

The control of the operation specifically described in “flow of processes of controller 3a” described below.

The display controller 33a receives detected values from the hygrometer 1a and the semiconductor gas sensor 2a from the detected value receiving section 31a, or the humidity and the ethanol concentration calculated by the calculating section 34a and instructs the display section 4a to display the detected values and the ethanol concentration on the display section 4a.

If the detected value is disclosed to the user, the user can check whether the alcohol detecting system 10a normally functions. The user can manipulate control conditions such as a detected temperature of the semiconductor gas sensor 2a based on the display data, for example, in order to adjust sensitivity of the semiconductor gas sensor 2a.

The display controller 33a may perform instruction so as to display only the ethanol concentration calculated based on the hygrometer 1a and the semiconductor gas sensor 2a. Otherwise, the display controller 33a may perform instruction so as to display a sobriety level determined from the calculated ethanol concentration.

The calculating section 34a calculates a detected value and a concentration of ethanol from the detected values of the hygrometer 1a and the semiconductor gas sensor 2a.

That is, the calculating section 34a excludes a detected value (detected value of humidity) of the detection target common to both of the hygrometer 1a and the semiconductor gas sensor 2a from the detected value from the semiconductor gas sensor 2a and sets the detected value of the detection target (detected value of ethanol), which is not common to the hygrometer 1a, of the semiconductor gas sensor 2a as the detected value from the semiconductor gas sensor 2a. The detected value is sent to the display controller 33a and the storage section 5a.

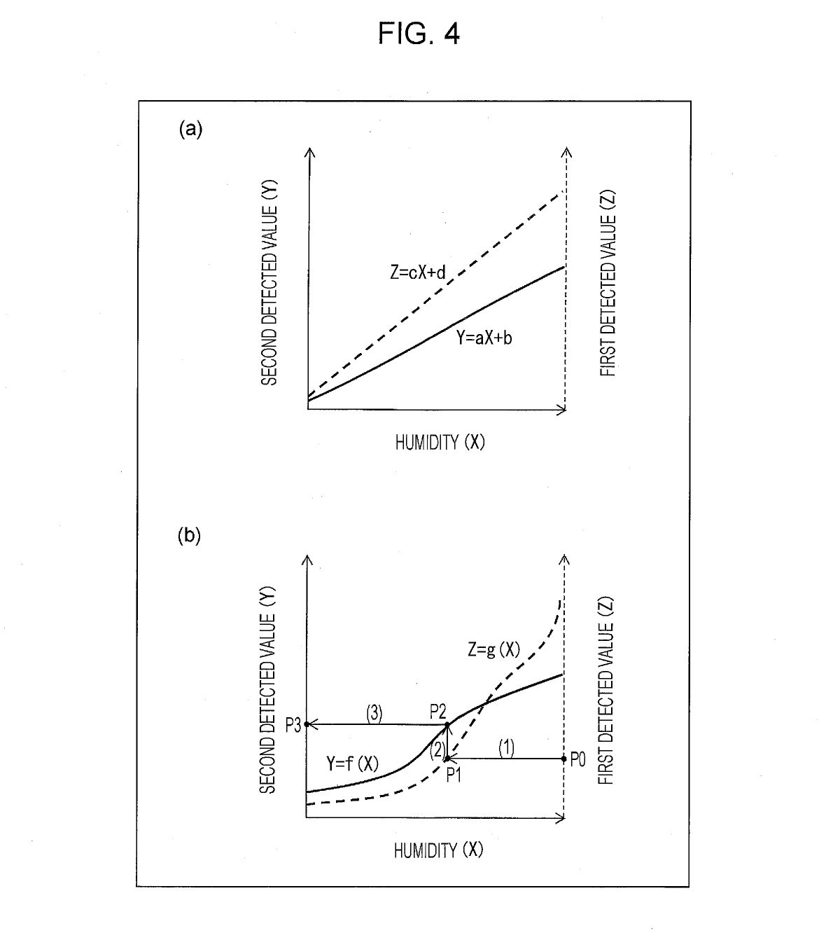

FIG. 4(a) is an example of a graph illustrating detected values of humidity in the hygrometer 1a and the semiconductor gas sensor 2a.

Here, the detected value from the hygrometer 1a (first detector) is indicated by Z=cX+d. The detected value from the semiconductor gas sensor 2a (second detector) is indicated by Y=aX+b.

If the value of Y is indicated with Z by using two equations above, Y=a(Z−d)/c+b can be indicated. Accordingly, if the value of Z can be indicated, the value of Y can be calculated. That is, a degree (detected value of the corresponding humidity in the semiconductor gas sensor 2a) in which the humidity influence on the detected value from the semiconductor gas sensor can be calculated from the detected value detected by a hygrometer in a specific humidity by using a relation expression between humidity and the detected value from the hygrometer 1a and a relation expression between humidity and the detected value from the semiconductor gas sensor 2a.

Subsequently, the calculating section 34a calculates a difference value between a detected value (sum of detected values of ethanol and humidity) measured in the semiconductor gas sensor 2a and the detected value of the humidity in the semiconductor gas sensor 2a calculated as described above. The detected value of ethanol can be calculated by calculating the difference value.

FIG. 4(b) is another example of a graph illustrating detected values with respect to the humidity of the hygrometer 1a and the semiconductor gas sensor 2a.

Here, the detected value from the hygrometer 1a (first detector) is indicated by Z=g(X). The detected value from the semiconductor gas sensor 2a (second detector) is indicated by Y=f(X).

As illustrated in FIG. 4(b), Z=g(X) or Y=f(X) are not simple functions such as a linear function.

The storage section 5a may store a graph of relation expression with humidity and detected values from the hygrometer 1a and humidity and a semiconductor gas sensor 2a as illustrated in FIG. 4(b).

The calculating section 34a calculates a degree (the detected value of the corresponding humidity in the semiconductor gas sensor 2a) in which specific humidity influences on the detected value in the semiconductor gas sensor 2a by using the corresponding table.

Specifically, the calculating section 34a illustrated in FIG. 4(b) specifics an intersection point P1 with Z=g(X) by moving a detected value P0 detected by the hygrometer 1a in an X axis in parallel. Subsequently, the calculating section 34a specifies an intersection point P2 with Y=f(X) by moving the point P1 in a Y axis in parallel. Subsequently, the calculating section 34a specifies an intersection point P3 with a Y axis by moving the intersection point P2 in an X axis in parallel. If the Y coordinate of the intersection point P3 is specified, degree (detected value in the corresponding humidity in the semiconductor gas sensor 2a) in which the corresponding humidity influence on the detected value from the semiconductor gas sensor is calculated from the detected value detected by the hygrometer 1a in specified humidity.

Subsequently, the detected value of ethanol in the semiconductor gas sensor 2a is calculated by calculating a difference value between the detected value measured in the semiconductor gas sensor 2a and the detected value of the humidity in the semiconductor gas sensor 2a calculated as described above.

The calculating section 34a may have a configuration of calculating an ethanol concentration from the corresponding detected value of ethanol.

The display section 4a displays the humidity, the ethanol concentration, and the like, according to the instruction of the display controller 33a.

The storage section 5a stores correction formulae and correction factors calculated by the calculating section 34a. The storage section 5a stores the detected value from the semiconductor gas sensor 2a and the detected value from the hygrometer 1a. The storage section 5a stores a control program or the like in the controller 3a.

(Flow of Processes of Controller 3a)

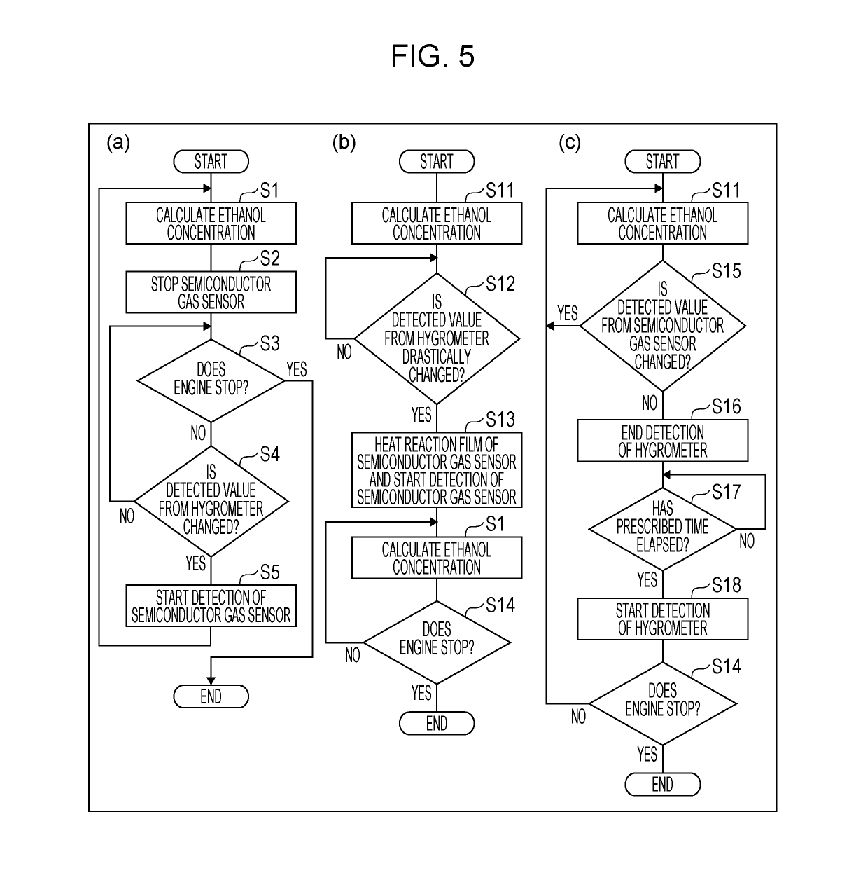

Subsequently, a flow of data processes performed by the controller 3a is described by using FIG. 5(a). FIG. 5(a) is a flow chart illustrating a flow of data processes performed by the controller 3a according to the embodiment.

The alcohol detecting system 10a starts an operation, for example, when a door of a vehicle opens, when a person sits in a driver's seat, or when an engine is started. That is, at this point of time, the hygrometer 1a and the semiconductor gas sensor 2a start detection. If the detected value receiving section 31a receives detected values from the hygrometer 1a and the semiconductor gas sensor 2a, the corresponding detected values are stored in the storage section 5a. The calculating section 34a reads detected values stored in the storage section 5a and calculates an ethanol concentration excluding humidity and influence on the humidity, according to the method described above (Step S1).

The sensor operation determining section 32a stops detection of the semiconductor gas sensor 2a after the calculation of the ethanol concentration (Step S2).

The sensor operation determining section 32a stops an operation of the alcohol detecting system 10a when a person disappears from a driver's seat or in a case where an engine stops (Step S3).

In the case of NO in Step S3, the hygrometer 1a performs detection operation continuously or in a prescribed time interval. The sensor operation determining section 32a monitors the detected value from the hygrometer 1a at a prescribed time interval and determines whether a change amount of the detected value from the hygrometer 1a is within a prescribed value (Step S4).

In a case where the sensor operation determining section 32a determines that a change amount of the detected value from the hygrometer 1a is within prescribed value (NO in Step S4), the sensor operation determining section 32a proceeds to Step S3 without starting an operation of the semiconductor gas sensor 2a.

In a case where the sensor operation determining section 32a determines that the change amount of the detected value from the hygrometer 1a is not within the prescribed value (YES in Step S4), the sensor operation determining section 32a instructs the start of detection of the semiconductor gas sensor 2a (Step S3). Thereafter, the process proceeds to Step S1.

It is assumed that, if a person in the vehicle drinks alcohol, humidity included in sweat or exhaled breath drastically changes, and thus the value detected by the hygrometer 1a drastically changes. Accordingly, it is possible to increase a life span of the semiconductor gas sensor 2a by operating the semiconductor gas sensor 2a only when the operation is necessary.

In a case where the sensor operation determining section 32a determines the change amount of the detected value from the hygrometer 1a is within the prescribed value (NO in Step S4), the process proceeds to Step S3.

### MODIFICATION EXAMPLE 1

Subsequently, a flow of other data processes is described by using FIG. 5(b).

The alcohol detecting system 10a starts an operation, for example, when a door of a vehicle opens, when a person sits in a driver's seat, or when an engine is started. That is, at this point of time, the hygrometer 1a and the semiconductor gas sensor 2a start detection. If the detected value receiving section 31a receives detected values from the hygrometer 1a and the semiconductor gas sensor 2a, the corresponding detected values are stored in the storage section 5a. The calculating section 34a reads detected values stored in the storage section 5a and calculates an ethanol concentration excluding humidity and influence on the humidity, according to the method described above (Step S11).

Subsequently to Step S11, the detected value receiving section 31a receives detected values from the hygrometer 1a and sends the detected values to the sensor operation determining section 32a. The sensor operation determining section 32a determines whether the detected value from the hygrometer 1a drastically changes (Step S12). In the corresponding determination, in a case where the change amount of the detected value from the hygrometer 1a per prescribed time changes twice or more, or in a case where a change amount of the detected value from the hygrometer 1a is a prescribed value (for example, in a case where a detected temperature in the semiconductor gas sensor 2a is set, a value of humidity that influences on the detected value from the semiconductor gas sensor 2a in the detected temperature) or greater, the sensor operation determining section 32a may determine that the detected value from the hygrometer 1a drastically changes.

In a case where the sensor operation determining section 32a determines that the detected value from the hygrometer 1a drastically changes (YES in Step S12), the sensor operation determining section 32a instructs the semiconductor gas sensor 2a to heat the reaction film. That is, by controlling the temperature of the reaction film of the semiconductor gas sensor 2a, performs control so that the semiconductor gas sensor 2a does not detect the humidity which is the detection target common to the hygrometer 1a (control the detection condition of the semiconductor gas sensor 2a) (Step S13).

For example, the heating of the corresponding reaction film may be performed until the detected value from the semiconductor gas sensor 2a becomes constant. The heating may be performed at a temperature and for a time in which the reaction film of the semiconductor gas sensor 2a is sufficiently refreshed. The semiconductor gas sensor 2a may restart the detection when the heating ends at a prescribed temperature or may perform detection while the reaction film is heated.

If a detected value receiving section 31b receives the detected value from the hygrometer 1a and the semiconductor gas sensor 2a, the detected value receiving section 31b sends the detected values from the hygrometer 1a and the semiconductor gas sensor 2a to the calculating section 34a. The calculating section 34a calculates the ethanol concentration from the detected values from the hygrometer 1a and the semiconductor gas sensor 2a (Step S1).

A configuration in which an operation of the alcohol detecting system 10a also stops when a person disappears from a driver's seat or when engine stops (Step S14) may be performed.

In the configuration above, the reaction film of the semiconductor gas sensor 2a can be refreshed when the humidity increases. Therefore, the semiconductor gas sensor 2a can perform detection in a state in which the influence of the humidity immediately after the refreshment is small. The water vapor concentration and the ethanol concentration can be independently (selectively) measured from the detected value from the hygrometer 1a and the detected value from the semiconductor gas sensor 2a.

Immediately after the reaction film of the semiconductor gas sensor 2a is heated, the detected value from the semiconductor gas sensor 2a does not receive an influence of the humidity and only detects ethanol. If time elapses from the heating of the reaction film, the reaction film of the semiconductor gas sensor 2a becomes colder, the detected value from the semiconductor gas sensor 2a receives an influence of the humidity.

At this point, if there is no change in the detected value from the hygrometer 1a, the humidity does not change. Accordingly, the detected value from the semiconductor gas sensor 2a in a case where the temperature of the reaction film of the semiconductor gas sensor 2a under constant humidity changes (decreases) can be measured.

In a case where there are changes in the detected value from the hygrometer 1a, the humidity can be calculated from the detected value from the hygrometer 1a. Accordingly, the detected value from the semiconductor gas sensor 2a in a case where the temperature of the reaction film of the semiconductor gas sensor 2a under specific humidity changes (decreases) can be measured.

That is, relationships among the humidity, the temperature of the semiconductor gas sensor 2a, and the detected value from the semiconductor gas sensor 2a can be analyzed by accumulating these measured data.

It is possible to determine whether the state of the reaction film of the semiconductor gas sensor 2a is normal (whether refreshment us sufficient or deterioration does not occur) by comparing the relationships among the detected value from the semiconductor gas sensor 2a after the reaction film of the semiconductor gas sensor 2a is heated, the temperature, and the humidity with the analyzed results.

After the detection start of the semiconductor gas sensor 2a, in a case where the sensor operation determining section 32a determines that the detected value from the hygrometer 1a changes to a prescribed value or greater, the sensor operation determining section 32a may perform instruction such that a temperature of the reaction film of the semiconductor gas sensor 2a is maintained to a temperature in which an influence of the humidity is sufficiently suppressed.

According to the configuration, the semiconductor gas sensor 2a can detect ethanol in a state in which influence of humidity is sufficiently suppressed. That is, the hygrometer 1a selectively performs detection and the semiconductor gas sensor 2a can selectively detects ethanol. The alcohol detecting system 10a according to the embodiment can suppress power consumption by comparing with a configuration in which the reaction film of the semiconductor gas sensor 2a is caused to be in a temperature in which influence of the humidity is always sufficiently suppressed. The alcohol detecting system 10a does not have a configuration in which the reaction film of the semiconductor gas sensor 2a is always heated at the time of detection. That is, the reaction film of the semiconductor gas sensor 2a has a configuration of being heated, if necessary. Therefore, the consumption of the heated reaction film can be reduced, and the life span of the semiconductor gas sensor 2a can be increased. Accordingly, the life span of the alcohol detecting system 10a can be increased.

According to the detected value from the hygrometer 1a, a configuration in which the detection of the semiconductor gas sensor 2a is started or stopped (ended) or the detection condition is controlled or a configuration in which detection of the hygrometer 1a starts or stops (ends) or the detection condition is controlled may be performed according to the detected value from the semiconductor gas sensor 2a.

### MODIFICATION EXAMPLE 2

Subsequently, a modification example of the flow of the data processes performed by the controller 3a is described by using FIG. 5(c). According to this modification example, the first detector is set as a semiconductor gas sensor, and the second detector is set as a digital hygrometer. The controller 3a determines operations of the first detector and the second detector based on the detected values from the semiconductor gas sensor which is the first detector and the digital hygrometer which is the second detector. FIG. 5(c) is a flow chart illustrating a flow of data processes performed by the controller 3a according to this modification example.

The alcohol detecting system 10a according to this modification example starts an operation when a door of a vehicle opens, when a person sits in a driver's seat, or when an engine is started. That is, at this point of time, the hygrometer and the semiconductor gas sensor start detection. If the detected value receiving section receives detected values from the hygrometer and the semiconductor gas sensor 2a, the corresponding detected values are stored in the storage section 5a. The calculating section 34a reads detected values stored in the storage section 5a and calculates an ethanol concentration excluding humidity and influence on the humidity, according to the method described above (Step S11).

The sensor operation determining section 32a determines whether the change amount of the detected value from the semiconductor gas sensor 2a is within the prescribed value (Step S15).

In a case where the sensor operation determining section 32a determines whether the change amount of the detected value from the semiconductor gas sensor 2a is within the prescribed value (is not changed) (NO in Step S15), the sensor operation determining section 32a instructs the hygrometer 1a to stop (end) detection (Step S16).

If the sensor operation determining section 32a instructs the hygrometer 1a to stop (end) the detection, the sensor operation determining section 32a determines whether prescribed time has elapsed from the corresponding instruction (Step S17). For example, the sensor operation determining section 32a refers to a timer section that can measure the elapsed time and performs the determination.

In a case where the sensor operation determining section 32a determines that prescribed time has elapsed (YES in Step S17), the sensor operation determining section 32a instructs the hygrometer 1a to start the detection (Step S18).

An operation of the alcohol detecting system 10a also stops when a person disappears from a driver's seat or in a case where engine stops (Step S14).

In a case where the sensor operation determining section 32a determines that the change amount of the detected value from the semiconductor gas sensor 2a is not within the prescribed value (changed) (YES in Step S15), the process proceeds to Step S11.

After the sensor operation determining section 32a instructs the hygrometer 1a to stop (end) the detection (Step S16), in a case where the sensor operation determining section 32a determines whether the change amount of the detected value from the semiconductor gas sensor 2a is within the prescribed value and determines the change amount of the detected value from the semiconductor gas sensor 2a is within the prescribed value, the operation of the hygrometer may restart.

In this manner, this modification example has a configuration in which the detected values from the semiconductor gas sensor that reacts with both of humidity and ethanol are monitored. Therefore, if the detected value from the semiconductor gas sensor is within a prescribed range, it is possible to determine that humidity is not changed, and the hygrometer can be operated when the hygrometer is necessary. Accordingly, it is possible to suppress the usage frequency of the hygrometer and thus it is possible to increase the life span of the hygrometer.

In the period of time of stopping the hygrometer 1a, the ethanol concentration is accurately measured without consideration and also the changes of the humidity and power consumption can be reduced by an amount of stopping the detection of the hygrometer 1a.

In the above, according to this embodiment and this modification example, the alcohol detecting system 10a is described as an example, but a methane/hydrogen sensor that is used in the fuel cell may be applied, instead of the semiconductor gas sensor 2a. According to the configuration, it is possible to realize the sensing system that can perform detection without receiving an influence of the humidity. Accordingly, fuel cells and vehicles to which the fuel cells are mounted can be operated with low power consumption and low running cost.

In a case where a detector that optically detects changes of the physical parameter of reaction film (oxide semiconductor and the like) that reacts with the detection target in the semiconductor gas sensor 2a is used, refreshment of the reaction film may be performed by photo irradiation.

### Embodiment 2

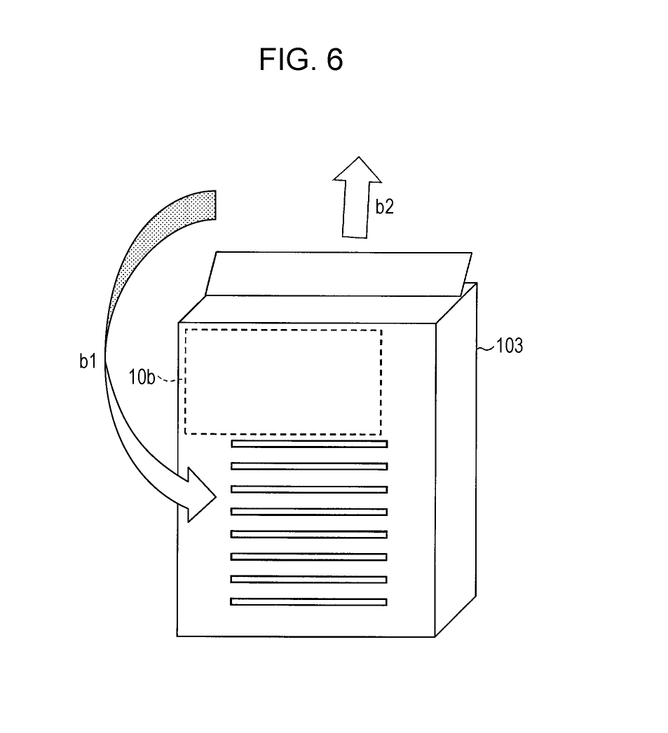

Another embodiment according to the invention is described below based on FIGS. 6 to 9. For convenience of explanation, members having the same functions as members described in the embodiment are denoted by the same reference numerals, and the descriptions thereof are omitted.

(External Appearance of Air Quality Monitoring System 10b)

An example of an external appearance of the sensing system according to the embodiment is described by using FIG. 6. The sensing system according to the embodiment is an air quality monitoring system 10b (sensing system). The detection purpose of the air quality monitoring system 10b is to obtain information relating to the air quality.

FIG. 6 is a diagram illustrating an example in which the air quality monitoring system 10b is mounted on an air cleaner 103. The air cleaner 103 sucks the air in a direction of a direction b1 and discharges the air in a direction of a direction b2. For example, the air quality monitoring system 10b may be included in an air conditioner (not illustrated).

In the air quality monitoring system 10b according to the embodiment, for example, a semiconductor gas sensor (first detector) 1b always detects a gas concentration of a volatile organic compound (VOC). If a detected value from a semiconductor gas sensor 1b is a prescribed value or greater, detection of a light absorbance-type gas sensor (second detector) 2b that detects the concentration of aldehyde-based gas is started.

The air quality monitoring system 10b independently (selectively) measures the detected value from the semiconductor gas sensor 1b and an aldehyde-based gas concentration and other gas concentrations from the light absorbance-type gas sensor 2b.

For example, the air quality monitoring system 10b may be mounted to an air purifier. For example, in a case where the air purifier cleans toluene and xylene having a heavy specific gravity, the air (toluene or xylene) close to a floor surface is circulated by causing exhaust air from the air purifier to be an air flow blowing diagonally forward and is and sucked. Meanwhile, in a case where the air cleaner cleans aldehyde-based gas having a light specific gravity and easily being collected at a ceiling, the air throughout the room is circulated by causing the exhaust air from the air purifier to be a vertical air flow and sucks the aldehyde-based gas.

(Main Configurations of Air Quality Monitoring System 10b)

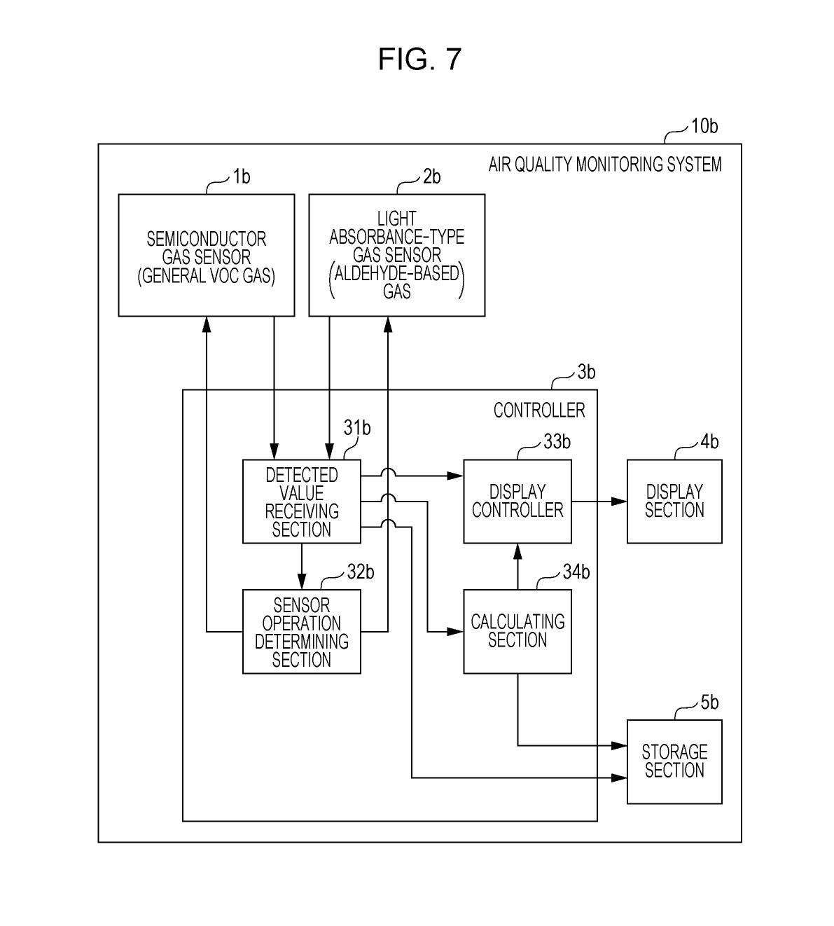

Subsequently, with reference to FIG. 7, main configurations of the air quality monitoring system 10b according to the embodiment are described. FIG. 7 is a functional block diagram schematically illustrating a configuration of the air quality monitoring system 10b. As illustrated in FIG. 7, the air quality monitoring system 10b includes the semiconductor gas sensor 1b, the light absorbance-type gas sensor 2b, a controller 3b, a display section 4b, and a storage section 5b.

(Semiconductor Gas Sensor 1b)

The semiconductor gas sensor 1b is a semiconductor gas sensor. The semiconductor gas sensor 1b detects general VOC gas as a detection target. The semiconductor gas sensor 1b sends the detected value to the controller 3b.

(Light Absorbance-Type Gas Sensor 2b)

The light absorbance-type gas sensor 2b is a light absorbance-type gas sensor. The light absorbance-type gas sensor 2b detects aldehyde-based gas as the detection target. The light absorbance-type gas sensor 2b includes a reaction chip of which color changes when aldehyde-based gas comes into contact with the reaction chip and a measuring apparatus that measures absorbance of the chip. The light absorbance-type gas sensor 2b sends the detected value to the controller 3b.

The detection target of the semiconductor gas sensor 1b and the detection target of the light absorbance-type gas sensor 2b are detection targets included in the first concept. The common detection target of the semiconductor gas sensor 1b and the light absorbance-type gas sensor 2b is aldehyde-based gas included in a subordinate concept of the first concept.

The controller 3b include the detected value receiving section 31b, a sensor operation determining section 32b, a display controller 33b, and a calculating section 34b.

The detected value receiving section 31b is the same as the detected value receiving section 31a described in Embodiment 1, and thus descriptions thereof are omitted.

The sensor operation determining section 32b determines detected values from the semiconductor gas sensor 1b and/or the light absorbance-type gas sensor 2b and controls of the operation of the semiconductor gas sensor 1b or the light absorbance-type gas sensor 2b.

The control of the operation is described in “flow of processes controller 3b” below.

The display controller 33b receives detected values from the semiconductor gas sensor 1b and the light absorbance-type gas sensor 2b from the detected value receiving section 31b or VOC gas concentration, aldehyde-based gas concentration calculated by the calculating section 34a, and the like and instructs the display section 4b to display the corresponding values.

If the detected values are disclosed to the user, the user can check whether the air quality monitoring system 10b normally functions.

The user can manipulate control conditions for temporarily stopping the detection or refreshing the reaction film based on the display data.

The display controller 33b perform instruction for displaying only the aldehyde-based gas concentration calculated from the detected value from the semiconductor gas sensor 1b and the light absorbance-type gas sensor 2b. Otherwise, the display controller 33b may perform instruction for displaying a cleanness level of the air determined from the calculated aldehyde-based gas concentration.

The calculating section 34b calculates the detected values and the concentrations of gas other than aldehyde-based gas from the detected values from the semiconductor gas sensor 1b and the light absorbance-type gas sensor 2b. The calculated value is sent to the display controller 33b and the storage section 5b.

The calculating section 34b may delete a detected value (detected value of aldehyde-based gas) of the detection target common to both of the light absorbance-type gas sensor 2b and the semiconductor gas sensor 1b from detected values from the semiconductor gas sensor 1b and set a detected value (detected value of gas other than aldehyde-based gas) of the detection target, which is not common to the light absorbance-type gas sensor 2b, of the semiconductor gas sensor 1b as the detected value from the semiconductor gas sensor 1b.

With respect to the calculation of the detected value of gas other than the aldehyde-based gas, a method described in the calculation of the detected value of ethanol from the calculating section 34a described in Embodiment 1 can be applied. For example, the detected value and the concentration of the gas other than the aldehyde-based gas may be calculated by substituting the detected value from the hygrometer 1a in the calculation of the detected value of ethanol described above to the detected value of the light absorbance-type gas sensor 2b and substituting the detected value of the semiconductor gas sensor 2a to the detected value of the semiconductor gas sensor 1b.

The display section 4b displays detected values, concentrations of aldehyde-based gas, and VOC gas and gas other than aldehyde-based gas in VOC gas, and the like, in response to the instruction of the display controller 33b.

The storage section 5b stores a correction formula, a correction factor, a graph, and the like calculated by the calculating section 34b. The storage section 5b stores a detected value of the light absorbance-type gas sensor 2b and a detected value of the semiconductor gas sensor 1b. The storage section 5b stores a control program executed by the controller 3b.

(Flow of Process of Controller 3b)

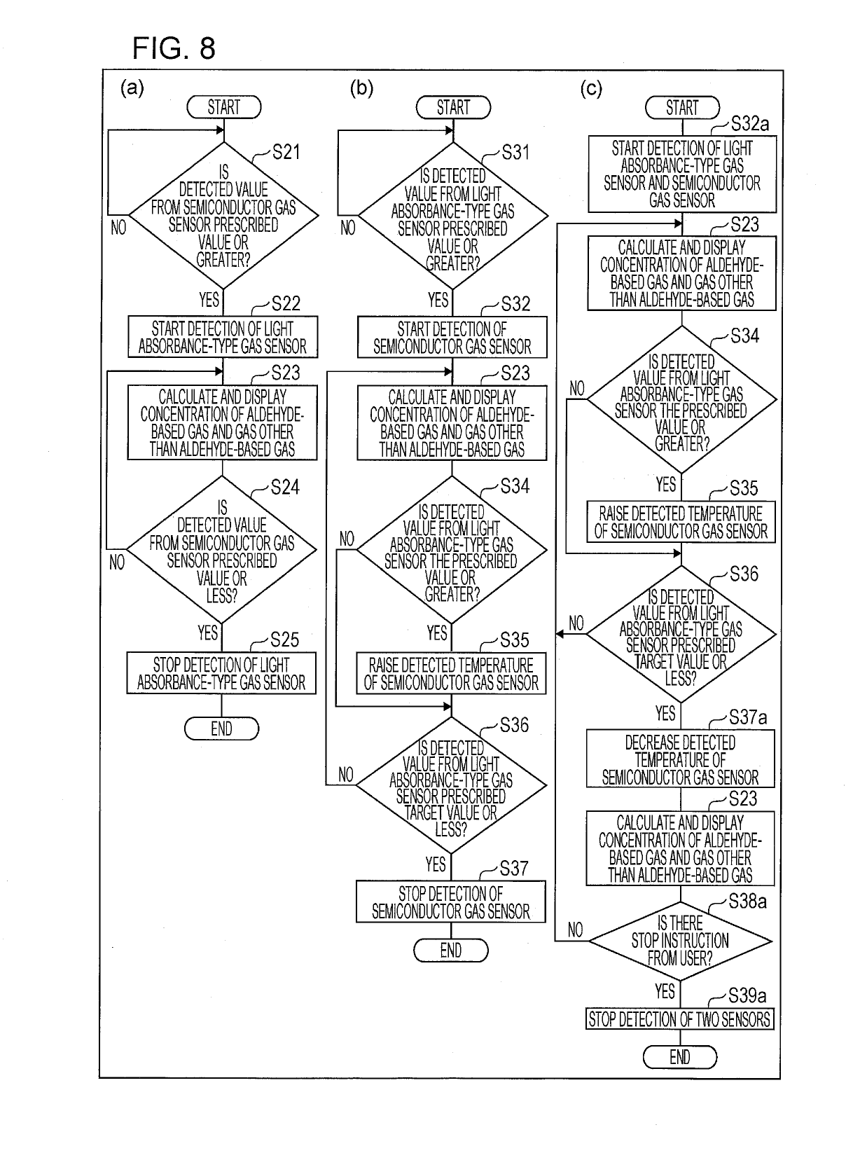

Subsequently, the flow of data processes performed by the controller 3b is described by using FIG. 8(a). FIG. 8(a) is a flow chart illustrating a flow of the data processes performed by the controller 3b according to the embodiment.

The detected value receiving section 31b receives the detected value from the semiconductor sensor 1b and sends the detected value to the sensor operation determining section 32b. The sensor operation determining section 32b determines whether the detected value from the semiconductor gas sensor 1b is the prescribed value or greater (Step S21).

For example, the corresponding prescribed value may be set as the detected value corresponding to the minimum concentration that damages health of the human body in any gas that can be detected by the semiconductor gas sensor 1b. The any gas may set as the most toxic gas among gas that can be detected by the semiconductor gas sensor 1b.

In a case where the sensor operation determining section 32b determines that the detected value from the semiconductor gas sensor 1b is the prescribed value or greater (YES in Step S21), the sensor operation determining section 32b instructs the light absorbance-type gas sensor 2b to start detection (Step S22).

If the detected value receiving section 31b receives the detected values from the semiconductor gas sensor 1b and the light absorbance-type gas sensor 2b, the detected values from the semiconductor gas sensor 1b and the light absorbance-type gas sensor 2b are sent to the calculating section 34b. The calculating section 34b calculates the concentrations of VOC gas, aldehyde-based gas, and gas other than aldehyde-based gas in VOC gas from the detected values from the semiconductor gas sensor 1b and the light absorbance-type gas sensor 2b and the concentration is displayed on the display section 4b (Step S23).

The sensor operation determining section 32b determines whether the detected value from the semiconductor gas sensor 1b is a prescribed target value or less (Step S24). In the corresponding step, the sensor operation determining section 32b monitors the detected values from the semiconductor gas sensor 1b at a prescribed time interval and determines whether a change amount of the detected value from the semiconductor gas sensor 1b is a prescribed value or less. The corresponding target value may be a target value configured in an apparatus to which the air quality monitoring system 10b is mounted.

In a case where the sensor operation determining section 32b determines that the detected value from the semiconductor gas sensor 1b is a prescribed target value or less (YES in Step S24), the sensor operation determining section 32b instructs the light absorbance-type gas sensor 2b to stop (end) the detection (Step S25).

In a case where the sensor operation determining section 32b does not determine that the detected value from the semiconductor gas sensor 1b is prescribed value or greater (NO in Step S21), the sensor operation determining section 32b receives the detected value from the semiconductor gas sensor 1b. Thereafter, the process proceeds to Step S21.

In a case where the sensor operation determining section 32b does not determine that the detected value from the semiconductor gas sensor 1b is within the prescribed target value or less (NO in Step S24), the process proceeds to Step S23.

For example, the light absorbance-type gas sensor 2b needs time for initialize (refresh) the reaction chip. In the configuration in which the reaction film is refreshed while the light absorbance-type gas sensor 2b does not perform detection, the detection of the light absorbance-type gas sensor 2b can be accurately performed at a desired timing (when the detected value from the semiconductor gas sensor 1b is a prescribed value or greater). Therefore, if the air quality monitoring system 10b according to the embodiment is mounted to an air conditioner or an air purifier, the air conditioner or the air purifier can be controlled according to accurate detection results of the air quality monitoring system 10b. Therefore, the corresponding air conditioner and the corresponding air purifier can effectively control the air quality in a room.

In a case where the reaction chip of the light absorbance-type gas sensor 2b is dealt as consumables, if the light absorbance-type gas sensor 2b is always detected, a lot of reaction chips are consumed.

The air quality monitoring system 10b can control the light absorbance-type gas sensor 2b so as to perform detection only when necessary according to the detected value from the semiconductor gas sensor 1b. Therefore, the life span of the reaction chip of the light absorbance-type gas sensor 2b increases, and thus the user does not have to frequently replace the reaction chips.

The calculating section 34b calculates the concentration VOC gas, the concentration of aldehyde-based gas, and the concentration of gas other than aldehyde-based gas in VOC gas from the detected values from the semiconductor gas sensor 1b and the light absorbance-type gas sensor 2b. Accordingly, the air quality monitoring system 10b can independently (selectively) measure VOC gas, aldehyde-based gas, and gas other than aldehyde-based gas in VOC gas.

If the detection speed of the semiconductor gas sensor 1b responses faster than the detection of the light absorbance-type gas sensor 2b, the followings can be said. That is, the response of the air quality monitoring system 10b is fast, compared with the air quality monitoring system in which the light absorbance-type gas sensor 2b always perform detection.

According to this embodiment, the air quality monitoring system 10b in which the detection target is set as gas is exemplified. However, a water quality monitoring system in which the detection target is set as components in a liquid may be used.

### MODIFICATION EXAMPLE

A modification example of the flow of the data processes performed by the controller 3b is described by using FIG. 8(b). The controller 3b according to this modification example controls the detection of the semiconductor gas sensor 1b according to the detected value from the light absorbance-type gas sensor 2b. FIG. 8(b) is a flow chart illustrating a flow of data processes performed by the controller 3b according to this modification example.

As illustrated in FIG. 8(b), the detected value receiving section 31b receives the detected value from the light absorbance-type gas sensor 2b and sends the detected value to the sensor operation determining section 32b. The sensor operation determining section 32b determines whether the detected value from the light absorbance-type gas sensor 2b is the prescribed value or greater (Step S31). For example, the corresponding prescribed value may be set as a detected value corresponding to the minimum concentration that damages health of the human body in any gas that can be detected by the light absorbance-type gas sensor 2b. The any gas may set as the most toxic gas among gas that can be detected by the light absorbance-type gas sensor 2b.

In a case where the sensor operation determining section 32b determines that the detected value from the light absorbance-type gas sensor 2b is the prescribed value or greater (YES in Step S31), the sensor operation determining section 32b instructs the semiconductor gas sensor 1b to start detection (Step S32).

Subsequently, the process proceeds to Step S23. Since Step S23 is described above, details descriptions thereof are omitted.

Subsequently to Step S23, the sensor operation determining section 32b determines that the detected value from the light absorbance-type gas sensor 2b is the prescribed value or greater (Step S34). In a case where the sensor operation determining section 32b determines that the detected value from the light absorbance-type gas sensor 2b is the prescribed value or greater (YES in Step S34), the sensor operation determining section 32b instructs the semiconductor gas sensor 1b to raise the temperature of the reaction film (Step S35).

Subsequently, the sensor operation determining section 32b determines that the detected value from the light absorbance-type gas sensor 2b is the prescribed target value or less (Step S36). In the corresponding step, the sensor operation determining section 32b may monitor the detected value from the light absorbance-type gas sensor 2b at a prescribed time interval and may determine whether the change amount of the detected value from the light absorbance-type gas sensor 2b is the prescribed value or less. The corresponding target value may be a target value that is configured in equipment to which the air quality monitoring system 10b is mounted.

In a case where the sensor operation determining section 32b determines whether the detected value from the light absorbance-type gas sensor 2b is the prescribed target value or less (YES in Step S36), the sensor operation determining section 32b instructs the semiconductor gas sensor 1b stop (end) detection (Step S37).

In a case where the sensor operation determining section 32b determines that the detected value from the light absorbance-type gas sensor 2b is not the prescribed value or greater (NO in Step S31), the sensor operation determining section 32b receives the detected value from the light absorbance-type gas sensor 2b and the process proceeds to Step S31.

In a case where the sensor operation determining section 32b determines that the detected value from the light absorbance-type gas sensor 2b is not the prescribed value or greater (NO in Step S34), the process proceeds to Step S36.

In a case where the sensor operation determining section 32b determines that the detected value from the light absorbance-type gas sensor 2b is not less than the prescribed target value (NO in Step S36), the process proceeds to Step S23.

Another data process according to this modification example is described by using FIG. 8(c). Only the differences from the modification example are described.

After the detection of the semiconductor gas sensor 1b and the detection of the light absorbance-type gas sensor 2b start (Step S32a), the process proceeds to Step S23. The processes from Step S23 to Step S36 are described above and thus the descriptions thereof are omitted.

In a case where the sensor operation determining section 32b determines that the detected value from the light absorbance-type gas sensor 2b is the prescribed target value or less (YES in Step S36), the sensor operation determining section 32b raises the detected temperature of the semiconductor gas sensor (Step S37a).

Subsequently, the process proceeds to Step S23. Step S23 is described above, and thus detailed descriptions thereof are omitted.

The sensor operation determining section 32b is determined whether there is an ending instruction from the user (Step S38a). In a case where there is an ending instruction from the user (YES in Step S38a), the detection of the semiconductor gas sensor 1b and the light absorbance-type gas sensor 2b is ended. In a case where there is not an ending instruction from the user (NO in Step S38a), the process proceeds to Step S23.

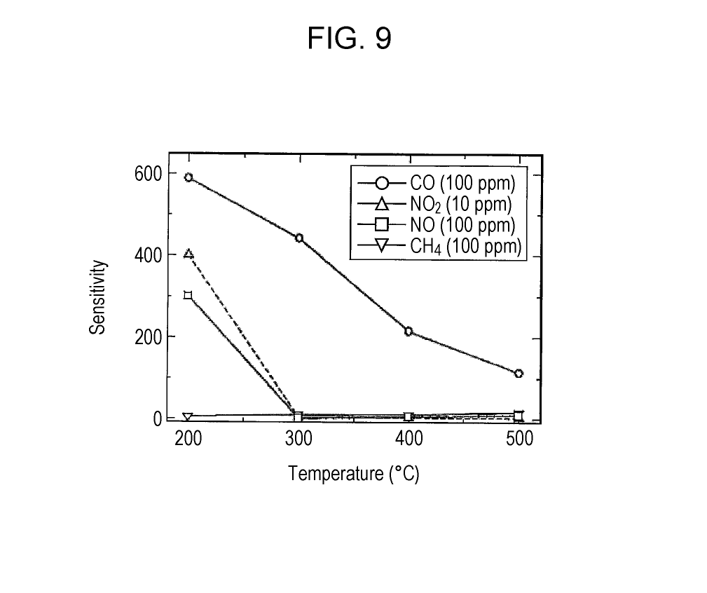

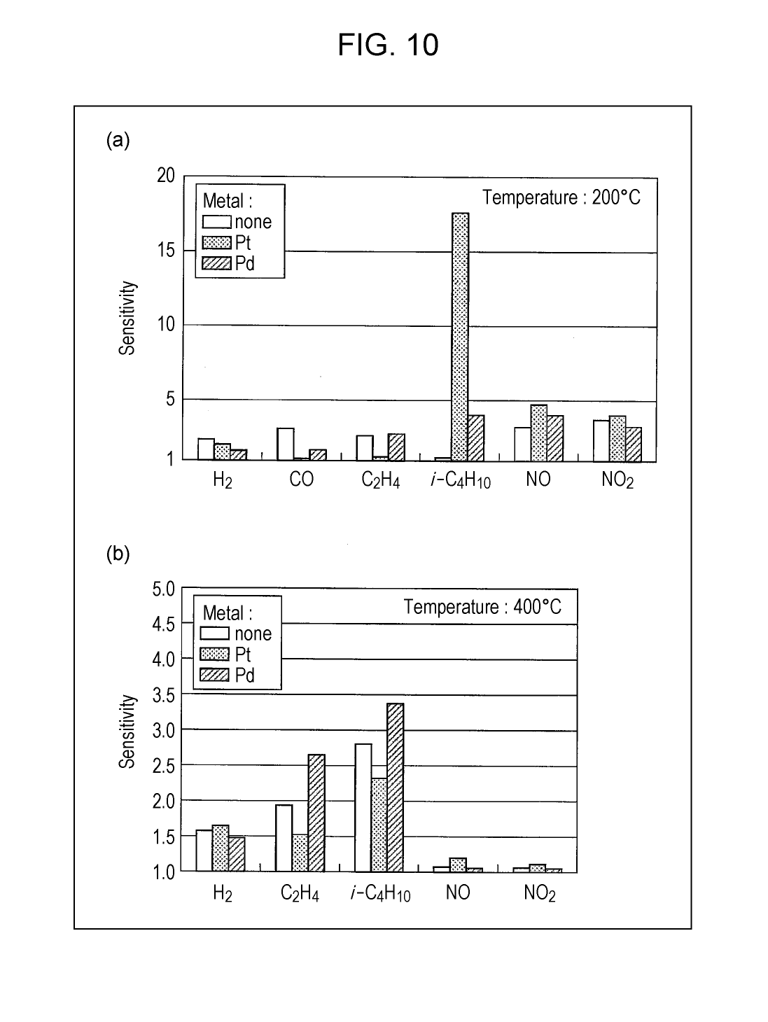

Here, the relationship with the detected temperature of the semiconductor gas sensor and the sensitivity to the detected gas is described. FIG. 9 is a graph illustrating sensitivity dependency to an operation temperature (detected temperature) of a gas sensor using a ZnO—SO2 composite thin film disclosed in NPL 1. FIG. 10(a) is a graph illustrating gas selectivity of the semiconductor gas sensor at 200° C. depending on whether Pt or Pd catalysts are combined disclosed in NPL 2. FIG. 10(b) is a graph illustrating gas selectivity of the semiconductor gas sensor at 200° C. depending on whether Pt or Pd catalysts are combined disclosed in NPL 2.

As illustrated in FIGS. 9 and 10, the semiconductor gas sensor changes sensitivity to the detected gas by the detected temperature. Accordingly, the selectivity of the detection target of the semiconductor gas sensor 1b can be changed by adjusting the temperature of the reaction film of the semiconductor gas sensor 1b and adjusting the detected temperature.

According to the data processes, in a case where the detected value from the light absorbance-type gas sensor 2b is the prescribed value or greater, the temperature of the reaction film of the semiconductor gas sensor 1b is raised. Therefore, in the same principle as the selectivity of the semiconductor gas sensor described above, the semiconductor gas sensor 1b can be controlled such that the semiconductor gas sensor 1b seldom detects aldehyde-based gas.

In this embodiment and the modification example, the sensor operation determining section 32b may stop detection of the light absorbance-type gas sensor 2b when the detected value from the semiconductor gas sensor 1b becomes a constant value. In this case, timing for starting the detection of the light absorbance-type gas sensor 2b may be determined according to the change amount of the detected value from the semiconductor gas sensor 1b. According to the detected value from the semiconductor gas sensor 1b, another operation condition (configured temperature, flow rate of detection target, applied voltage, and the like) of the light absorbance-type gas sensor 2b may be determined.

The operation condition influences on the detection sensitivity, the life span, and the power consumption of the light absorbance-type gas sensor 2b and the like. The air quality monitoring system 10b determines these operation conditions of the light absorbance-type gas sensor 2b according to the detected value from the semiconductor gas sensor 1b. Therefore, in view of detection sensitivity of the light absorbance-type gas sensor 2b, a usage period of time of the light absorbance-type gas sensor 2b, a period of time until refreshment, a power consumption of the light absorbance-type gas sensor 2b, a running cost of the light absorbance-type gas sensor 2b, and reduction of the consumption of the light absorbance-type gas sensor 2b, the suitable detection condition of the light absorbance-type gas sensor 2b can be configured.

### Embodiment 3

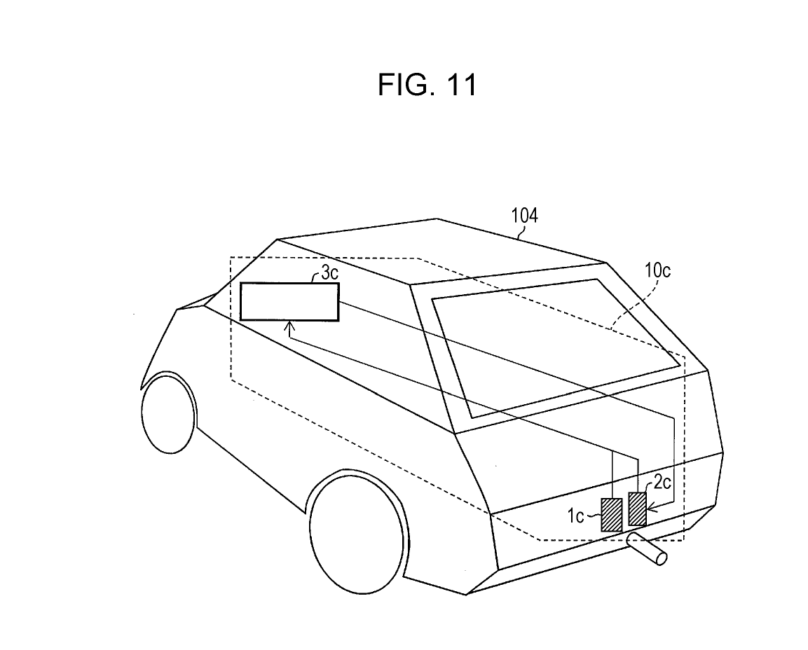

Another embodiment of the invention is described below based on FIGS. 11 to 13. For convenience of explanation, members having the same functions as members described in the embodiment are denoted by the same reference numerals, and the descriptions thereof are omitted.

(External Appearance of Gas Sensing System 10c)

An example of an external appearance of the sensing system according to the embodiment is described by using FIG. 11. The sensing system according to the embodiment is a gas sensing system 10c (gas sensing system). FIG. 11 is a diagram illustrating an example in which the gas sensing system 10c is mounted on an automobile 104. That is, the detection purpose of the gas sensing system 10c is to obtain information relating to the exhaust air gas concentration of the automobile 104.

In the gas sensing system 10c according to the embodiment, for example, a first semiconductor gas sensor (first detector) 1c of which the detection condition is configured as 200° C. always detect CO, NO, and NO2 gas concentrations, and if the detected value from the first semiconductor gas sensor 1c is the prescribed value or greater, the detection of a second semiconductor gas sensor 2c (second detector) of which the detection condition for detecting CO is configured as 400° C. starts.

That is, the gas sensing system 10c according to the embodiment has a configuration in which the detectors having the same detection principle detect the detection targets by the different detection condition. For example, as illustrated in FIG. 9, in the gas sensor using a ZnO—SO2 composite thin film, sensitivity dependency of the detection target varies depending on the operation temperature (detected temperature).

If it is determined that the detected value of CO from the gas sensing system 10c from the detected value from the second semiconductor gas sensor 2c is high, feed back is performed to the generation source of the detected gas. For example, if the detected gas having a high detected value is CO, the possibility of incomplete combustion of fuel is considered. The gas sensing system 10c sends the information illustrating that the CO concentration is high to a combustion controlling device 20 that controls the combustion of fuel. Accordingly, the combustion controlling device 20 can perform the control of diluting the fuel according to the corresponding information.

(Main Configurations of Gas Sensing System 10c)

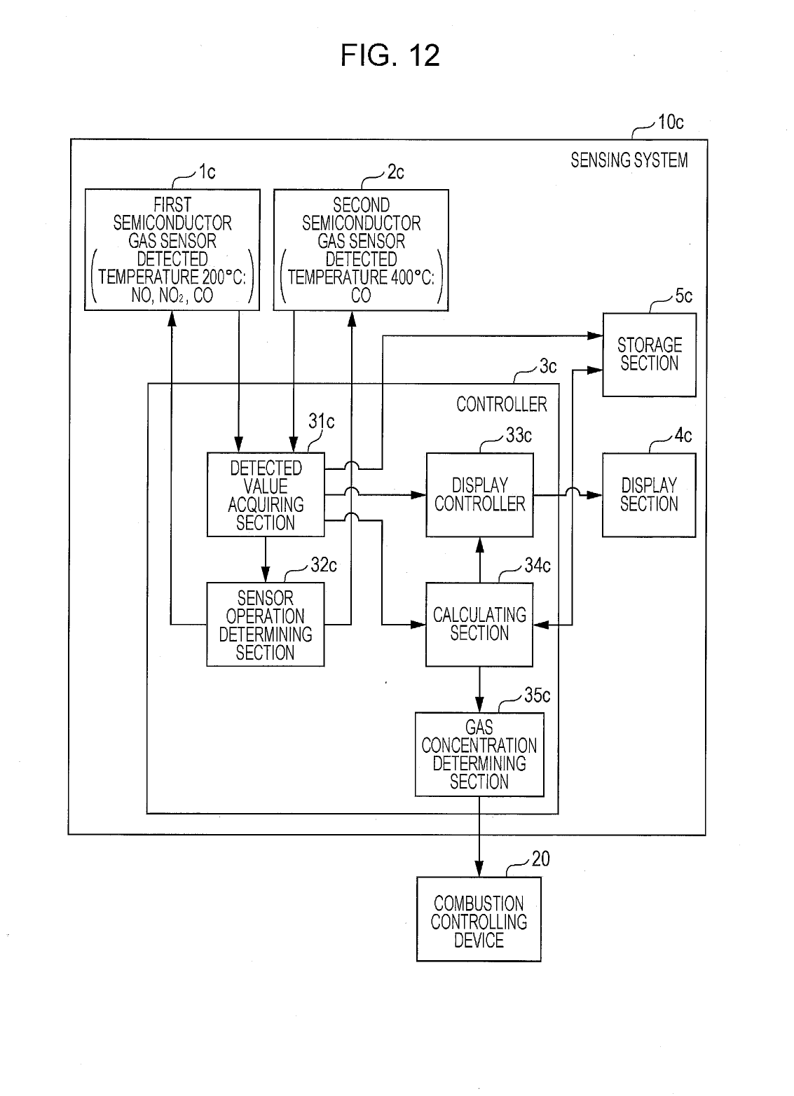

Subsequently, with reference to FIG. 12, main configurations of the gas sensing system 10c according to the embodiment are described. FIG. 12 is a functional block diagram schematically illustrating a configuration of the gas sensing system 10c. As illustrated in FIG. 12, the gas sensing system 10c includes the first semiconductor gas sensor 1c, the second semiconductor gas sensor 2c, a controller 3c, a display section 4c, and a storage section 5c.

(First Semiconductor Gas Sensor 1c)

The first semiconductor gas sensor 1c is a semiconductor gas sensor of which the detection condition is configured as 200° C. As illustrated in FIG. 12, the first semiconductor gas sensor 1c sets NO, NO2, and CO as detection targets. The first semiconductor gas sensor 1c sends the detected values to the controller 3c.

(Second Semiconductor Gas Sensor 2c)

The second semiconductor gas sensor 2c is a semiconductor gas sensor of which the detection condition is configured as 400° C. As illustrated in FIG. 12, the second semiconductor gas sensor 2c sets CO as a detection target. The second semiconductor gas sensor 2c sends the detected value to the controller 3c.

The detection target of the first semiconductor gas sensor 1c and the detection target of the second semiconductor gas sensor 2c are detection targets included in the first concept which is gas. The detection target common to the detection target of the first semiconductor gas sensor 1c and the detection target of the second semiconductor gas sensor 2c is CO included in the subordinate concept of the first concept.

The controller 3c includes a detected value acquiring section 31c, the sensor operation determining section 32c, the display controller 33c, a calculating section 34c, and a gas concentration determining section 35c.

The detected value acquiring section 31c receives the detected values from the first semiconductor gas sensor 1c and the second semiconductor gas sensor 2c, and sends the detected values to a sensor operation determining section 32c, a display controller 33c, the calculating section 34c, the gas concentration determining section 35c, and the storage section 5c.

The sensor operation determining section 32c determines detected values from the first semiconductor gas sensor 1c and/or the second semiconductor gas sensor 2c and controls an operation of the first semiconductor gas sensor 1c or the second semiconductor gas sensor 2c.

The control of the operation is specifically described in “flow of processes of controller 3c”.

The display controller 33c receives detected values from the first semiconductor gas sensor 1c and the second semiconductor gas sensor 2c from the detected value acquiring section 31c or the CO gas concentration calculated by the calculating section 34c and instructs the display section 4c to display the corresponding value.

If the detected value is disclosed to the user, the user can check whether the gas sensing system 10c normally functions.

The user can manipulate control conditions of an automobile such as accelerator and brake manipulation based on the display data.

The display controller 33c may display only the CO concentration and may display the combustion state determined from the CO concentration.

The calculating section 34c calculates the detected value of gas other than CO in the gas detected by the first semiconductor gas sensor 1c and the CO concentration from the detected value from the second semiconductor gas sensor 2c. The calculated value is sent to the gas concentration determining section 35c, the display controller 33c, and the storage section 5c.

The calculation section 34c may exclude a detected value (detected values of CO concentration) of the detection target common to both of the second semiconductor gas sensor 2c and the first semiconductor gas sensor 1c from the detected value from the first semiconductor gas sensor 1c and set a detected value (detected value of gas other than CO) of the detection target, which is not common to the second semiconductor gas sensor 2c, of the first semiconductor gas sensor 1c as the detected value from the semiconductor gas sensor 1c.

The gas concentration determining section 35c determines whether the CO concentration received from the calculating section 34c is the prescribed value or greater. The gas concentration determining section 35c may determine whether the detected value from the second semiconductor gas sensor 2c is the prescribed value or greater. If the calculated value or the detected value is the prescribed value or greater, the high concentration CO information indicating that CO is higher than the prescribed concentration is sent to the combustion controlling device 20.

The display section 4c displays the detected values from the first semiconductor gas sensor 1c and the second semiconductor gas sensor 2c and the concentration calculated from the corresponding detected values according to the instruction of the display controller 33c.

The storage section 5c stores the correction formula, the correction factor, the graph, and the like that are used by the calculating section 34c in calculation. The storage section 5c stores the detected values from the first semiconductor gas sensor 1c, the second semiconductor gas sensor 2c, and the like. The storage section 5c stores the control programs executed by the controller 3c.

(Flow of Processes of Controller 3c)

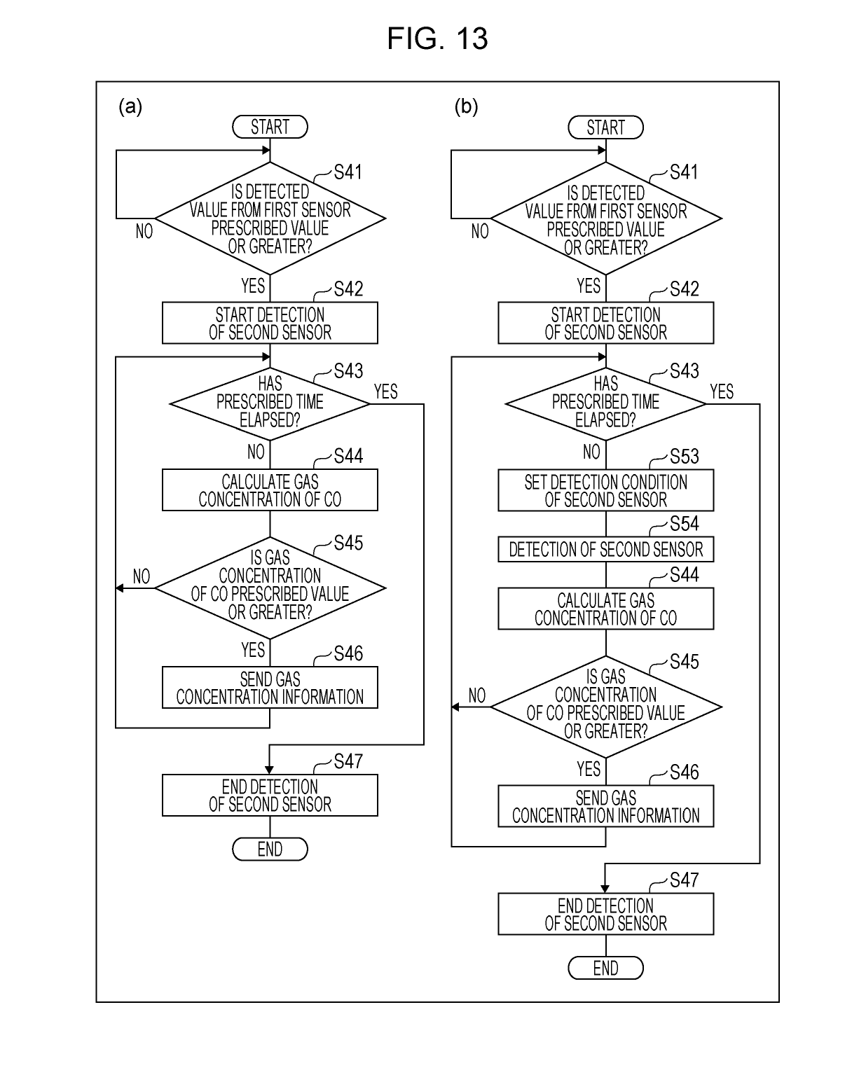

Subsequently, the flow of the data processes performed by the controller 3c is described by using FIG. 13(a). FIG. 13(a) is a flow chart illustrating a flow of data processes performed by the controller 3c according to the embodiment.

The detected value acquiring section 31c receives the detected values from the first semiconductor gas sensor 1c and sends the detected values to the sensor operation determining section 32c. The sensor operation determining section 32c determines whether the detected value from the first semiconductor gas sensor 1c is the prescribed value or greater (Step S41).

For example, the corresponding prescribed value is the detected value corresponding to the minimum concentration in which CO damages health of the human body.

In a case where the sensor operation determining section 32c determines that the detected value from the first semiconductor gas sensor 1c is a prescribed value or greater (YES in Step S41), the sensor operation determining section 32c instructs the second semiconductor gas sensor 2c to start detection (Step S42).

In Step S42, if the sensor operation determining section 32c instructs the second semiconductor gas sensor 2c to start detection, the sensor operation determining section 32c determines that prescribed time has elapsed from the corresponding instruction (Step S43). For example, the sensor operation determining section 32c may perform the determination with reference to the timer section that can measure the elapsed time.

The prescribed time is equal to or longer than the time in which an operation of the second semiconductor gas sensor 2c becomes stable, and the generation of CO gas can be accurately detected. For example, in a case where the corresponding prescribed time is configured, the corresponding prescribed time can be configured with reference to the detection result of the CO gas.

The prescribed time is a sufficient time that can detect the generation of the CO gas in which the concentration of CO gas becomes stable, the average value of the change can be determined, and the like.

The prescribed time is the time in which the power consumption of the second semiconductor gas sensor 2c is suppressed as much as possible.

In a case where the sensor operation determining section 32c determines that the prescribed time has not elapsed from the instruction of the detection start of the second semiconductor gas sensor 2c (NO in Step S43), the sensor operation determining section 32c does not instruct the second semiconductor gas sensor 2c to stop (end) the detection.

Subsequently, the calculating section 34c calculates the concentrations of CO and gas other than CO from the detected values from the first semiconductor gas sensor 1c and the second semiconductor gas sensor 2c received from the detected value acquiring section 31c. The calculating section 34c sends the calculated concentrations of CO and gas other than CO to the gas concentration determining section 35c (Step S44).

The gas concentration determining section 35c determines whether the concentration of the received CO is prescribed value or greater (Step S45).

In a case where the gas concentration determining section 35c determines that the received concentration of CO is a prescribed value or greater (YES in Step S45), the gas concentration determining section 35c sends the high concentration CO information to the combustion controlling device 20 (Step S46). Thereafter, the process proceeds to Step S43.

In a case where the sensor operation determining section 32c determines that the detected value from the first semiconductor gas sensor 1c is not the prescribed value or greater (NO in Step S41), the detected values are received from the first semiconductor gas sensor 1c and the process proceeds to Step S41.

In a case where the gas concentration determining section 35c determines that the received concentration of CO is not the prescribed value or greater (NO in Step S45), the process proceeds to Step S43.

In a case where the sensor operation determining section 32c determines that the prescribed time has elapsed from the start instruction of the second semiconductor gas sensor 2c (YES in Step S43), the sensor operation determining section 32c instructs the second semiconductor gas sensor 2c to stop (end) the detection (Step S47).

According to the configuration above, the first semiconductor gas sensor 1c and the second semiconductor gas sensor 2c are semiconductor gas sensors having the same detection principles. Accordingly, the process amounts of the control processes of two detectors can be reduced. Therefore, the controller 3c can be formed with the simple configuration.

If both of the first semiconductor gas sensor 1c and the second semiconductor gas sensor 2c always perform detection, power consumption is applied, and the first semiconductor gas sensor 1c and the second semiconductor gas sensor 2c are extremely consumed and thus the life spans thereof are shortened.

The gas sensing system 10c starts selective detection of the second semiconductor gas sensor 2c for detecting specific gas according to the detected values to the plurality types of gas of the first semiconductor gas sensor 1c.

Accordingly, the selective detection of the detection target and the realization of the gas sensing system 10c in which the energy saving and a long life span are compatible with each other becomes possible.

If the first semiconductor gas sensor 1c responses faster than the second semiconductor gas sensor 2c, the following can be said. That is, the response of the gas sensing system 10c is fast, compared with the gas sensing system in which the second semiconductor gas sensor 2c always perform detection.

### MODIFICATION EXAMPLE

Subsequently, a modification example of the flow of the data processes performed by the controller 3c is described by using FIG. 13(b). The controller 3c according to this modification example starts the detection of the second semiconductor gas sensor 2c according to the detected value from the first semiconductor gas sensor 1c and configures detection condition of the second semiconductor gas sensor 2c according to the detected value from the first semiconductor gas sensor 1c and/or the second semiconductor gas sensor 2c. FIG. 13(b) is a flow chart illustrating a flow of data processes performed by the controller 3b according to this modification example.

Steps S41, S42, and S43 are described above and thus the descriptions thereof are omitted.

As illustrated in FIG. 13(b), in a case where the sensor operation determining section 32c determines that the prescribed time has not elapsed from the instruction of the detection start of the second semiconductor gas sensor 2c (NO in Step S43), the sensor operation determining section 32c does not instruct the second semiconductor gas sensor 2c to stop (end) the detection the process proceeds to Step S53.

The sensor operation determining section 32c configures the detection condition of the second semiconductor gas sensor 2c according to the detected value from the second semiconductor gas sensor 2c received from the detected value acquiring section 31c. The sensor operation determining section 32c instructs the second semiconductor gas sensor 2c to detect the configured detection condition (Step S53).

Here, the detection condition of the second semiconductor gas sensor 2c configured by the sensor operation determining section 32c is described.

For example, in a case where the sensor operation determining section 32c determines that the concentration of gas other than CO is sufficiently low with respect to the CO concentration (for example, one tenth of CO concentration), the temperature of the reaction film of the second semiconductor gas sensor 2c is adjusted in order to raise the detection sensitivity of CO. For example, as illustrated in FIG. 9, detection sensitivity with respect to CO in a case where the reaction film is 300° C. is high, compared with a case where the reaction film of the second semiconductor gas sensor 2c is 400° C. Also in a case where the reaction film is 300° C., specificity of the detection with respect to CO can be maintained. Accordingly, the sensor operation determining section 32c may perform instruction such that the reaction film of the second semiconductor gas sensor 2c becomes 300° C.

If the sensor operation determining section 32c determines that the detected value from the second semiconductor gas sensor 2c is low, the detection time is configured to be long, and if the sensor operation determining section 32c determines that the detected value from the second semiconductor gas sensor 2c is high, the detection time is configured to be short.

The second semiconductor gas sensor 2c receives the instruction of the detection condition and performs detection in the corresponding configured detection condition (Step S54). Thereafter, the process proceeds to Step S44.

Steps S44, S45, S46, and S47 are described above, and thus the descriptions thereof are omitted.

Other operation conditions (configured temperature flow, rate of detection target, applied voltage, and the like) of the second semiconductor gas sensor 2c may be determined according to the detected value from the first semiconductor gas sensor 1c.

The operation conditions influence on the detection sensitivity, a life span, and a power consumption of the second semiconductor or gas sensor 2c and the like.

The gas sensing system 10c determines these operation conditions of the second semiconductor gas sensor 2c according to the detected value from the first semiconductor gas sensor 1c. Therefore, in view of the detection sensitivity of the second semiconductor gas sensor 2c, a period of time to the refreshment in the second semiconductor gas sensor 2c, a power consumption of the second semiconductor gas sensor 2c, a running cost of the second semiconductor gas sensor 2c, and consumption suppression of the second semiconductor gas sensor 2c, the suitable detection condition of the second semiconductor gas sensor 2c can be configured.

Particularly, in a case where the second semiconductor gas sensor 2c has the characteristics in that the life span is short, the power consumption is great, the running cost is high, a consumption section is included, and initialization (refreshment) is difficult, and the like, compared with that of the first semiconductor gas sensor 1c, the detection of the second semiconductor gas sensor 2c can be controlled in the gas sensing system 10c in the detection condition in which these disadvantages of the second semiconductor gas sensor 2c are suppressed.

According to the detected value from the second semiconductor gas sensor 2c received from the detected value acquiring section 31c, the sensor operation determining section 32c perform instruction such that the temperature of the reaction film of the second semiconductor gas sensor 2c is decreased from 400° C. to 300° C. Accordingly, as described above, while specificity of the detection to CO is maintained, detection sensitivity to CO can be raised. It is possible to reduce the power consumption by decreasing the detected temperature of the second semiconductor gas sensor 2c.

In this embodiment and the modification example, a gas sensing system for detecting CO is exemplified, but the target of the gas sensing system is not limited to CO. It is possible to realize the gas sensing system causing gas other than CO to be a target, for example, by the types of the reaction films of the first semiconductor gas sensor 1c and the second semiconductor gas sensor 2c, and detection conditions.

According to this embodiment, the gas sensing system 10c that detects the exhaust air gas of the automobile is exemplified, but the gas sensing system 10c can be applied to a system that detects organic gas such as ethylene, mercaptan, and the like generated from food in a refrigerator.

According to this embodiment, the gas sensing system in which the detection target is set as gas is exemplified, but the invention may be applied to a water quality monitoring system in which the detection target is set as components in the liquid.

In Embodiments 1, 2, and 3, the following configurations can be applied to the heating of the reaction film of the semiconductor gas sensor.

That is, in the semiconductor gas sensor that optically detects the changes of the physical parameter of the reaction film formed by the oxide semiconductor and the like, in a case where the reaction film is heated by light, the detection condition can be caused to be the light intensity that heats the reaction film.

### Embodiment 4

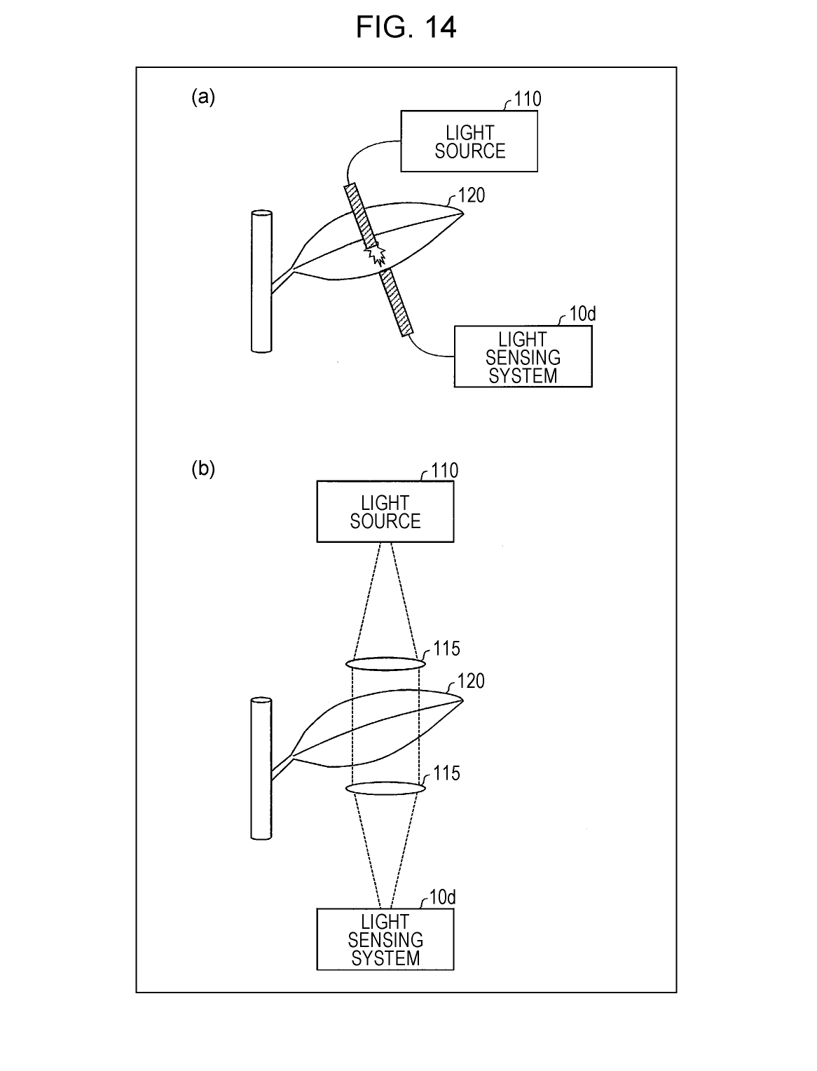

The embodiment of the invention is described below based on FIGS. 14 to 16. For convenience of explanation, members having the same functions as members described in the embodiment are denoted by the same reference numerals, and the descriptions thereof are omitted.

(External Appearance of Light Sensing System 10d)

An example of the external appearance of the sensing system according to the embodiment is described by using FIGS. 14(a) and 14(b). The sensing system according to the embodiment is a light sensing system 10d (sensing system) of which the detection purpose is to obtain information on existence or a state of a substance from the penetration, reflection, absorption, diffusion, and a light emission spectrum of light.

FIGS. 14(a) and 14(b) are diagrams illustrating external appearances of the light sensing system 10d. As illustrated in FIG. 14(a), for example, the light sensing system 10d irradiates a leaf 120 with light from a light source 110 by using an optical fiber. The light sensing system 10d detects light that penetrates the leaf 120 (penetrating light).

As an example of the light sensing system 10d, as illustrated in FIG. 14(b), the light sensing system 10d irradiates the leaf 120 with light from the light source 110 by using a lens 115. The light sensing system 10d detects light that penetrates the leaf 120 by using the lens 115.

The state of the leaf 120 can be analyzed by the light sensing system 10d.

The configuration in which the light sensing system 10d detects penetrating light is exemplified. However, a configuration in which the light sensing system 10d detects reflected or diffused light may be applied.

(Main Configurations of Light Sensing System 10d)

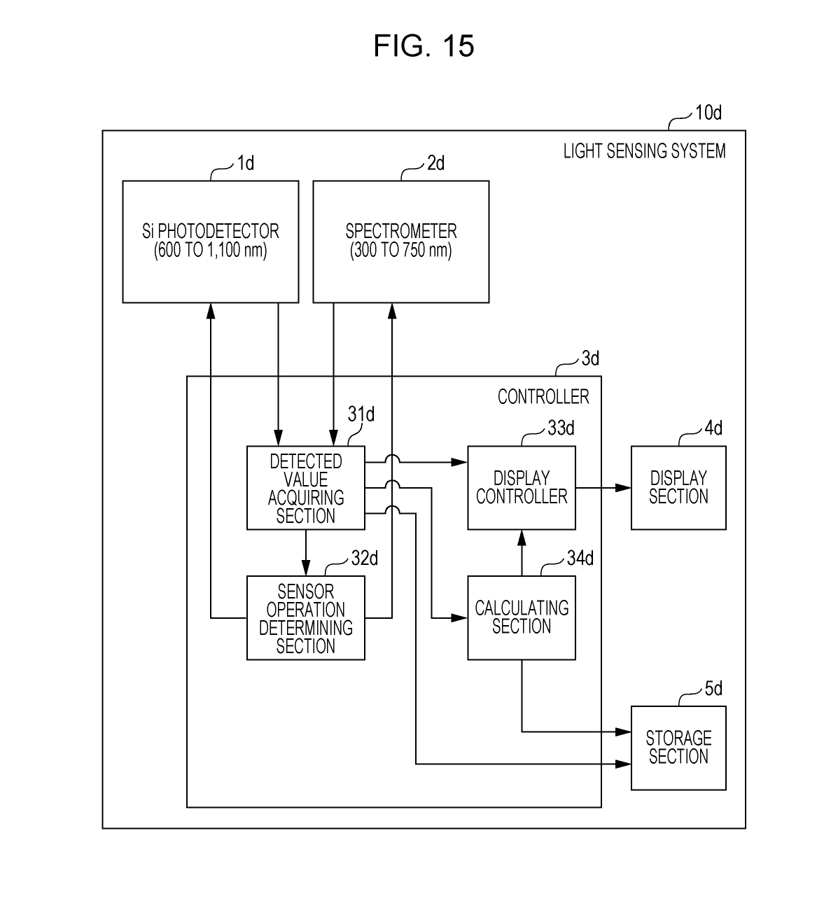

Subsequently, with reference to FIG. 15, main configurations of the light sensing system 10d according to the embodiment are described. FIG. 15 is a functional block diagram schematically illustrating a configuration of the light sensing system 10d. As illustrated in FIG. 15, the light sensing system 10d includes a Si photodetector (first detector) 1d, a spectrometer (second detector) 2d, a controller 3d, a display section 4d, and a storage section 5d.

(Si Photodetector 1d)

The Si photodetector 1d detects light in a red to infrared wavelength range (for example, 600 nm to 1,100 nm), as the detection target. The Si photodetector 1d sends the detected value to the controller 3d.

(Spectrometer 2d)

The spectrometer 2d detects light in an ultraviolet to near infrared wavelength range (for example, 300 nm to 750 nm) as the detection target. The spectrometer 2d sends the detected value to the controller 3d.

Here, the detection target of the Si photodetector 1d and the detection target of the spectrometer 2d are detection targets included in the first concept that is light intensity. The detection target common to the Si photodetector 1d and the spectrometer 2d is light intensity in a wavelength of 600 nm to 750 nm included in the subordinate concept of the first concept.

(Controller 3d)

The controller 3d includes a detected value acquiring section 31d, a sensor operation determining section 32d, a display controller 33d, and a calculating section 34d.

The detected value acquiring section 31d receives the detected value from the Si photodetector 1d and the spectrometer 2d and sends the sensor operation determining section 32d, the display controller 33d, the calculating section 34d, and the storage section 5d.

The sensor operation determining section 32d determines the detected values from the Si photodetector 1d and/or the spectrometer 2d and controls the detection operations of the Si photodetector 1d or the spectrometer 2d.

The control of the detection operation is specifically described below in “flow of processes of controller 3d”.

The display controller 33d receives the detected values from the Si photodetector 1d and the spectrometer 2d from the detected value acquiring section 31d or the light intensity in a wavelength calculated by the calculating section 34d and instructs the display section 4d to display the detected value and the light intensities of the respective wavelengths on the display section 4d.

If the detected value and the light intensity in the respective wavelengths are displayed on the display section 4d, the user can look at the corresponding display section 4d, so as to check whether the light sensing system 10d normally functions. The user can manipulate the control conditions such as a wavelength range of light to be measured, for example, based on the display data (detected value and the light intensity in the respective wavelengths) displayed on the display section 4d.

The display controller 33d may instruct the display section 4d to display the light intensity calculated from the detected values from the Si photodetector 1d and the spectrometer 2d. The display controller 33d may instruct the display section 4d to display a growth state or a moisture amount of the leaf determined from the detected values from the Si photodetector 1d and the spectrometer 2d.

The calculating section 34d calculates the detected value of the light intensity in a wavelength of 750 to 1,100 nm from the detected values from the Si photodetector 1d and the spectrometer 2d. The calculated values are sent to the display controller 33d and the storage section 5d.

The calculating section 34d may exclude a detected values (600 to 750 nm) of the detection target common to both of the Si photodetector 1d and the spectrometer 2d from the detected values of the Si photodetector 1d and set a detected value (detected value of 750 to 1,100 nm) from the detection target, which is not common to the spectrometer 2d, of the Si photodetector 1d as the detected value from the Si photodetector 1d.

In the calculation of the detected value of the light intensity in a wavelength of 750 to 1,100 nm, the calculation method described in the calculation of the detected value of ethanol of the calculating section 34a described in Embodiment 1 can be applied. For example, the integral detected value of the light intensity and the light intensity at a wavelength of 750 to 1,100 nm may be calculated by substituting the detected value from the hygrometer 1a in the calculation of the detected value of ethanol of Embodiment 1 to a detected value integrated with 600 nm to 750 nm of the spectrometer 2d and substituting the detected value from the semiconductor gas sensor 2a of Embodiment 1 to the detected value from the Si photodetector 1d.

The “integrated detected value” and the “integral detected value” are integral value of the light intensity in a certain wavelength range in a case where a horizontal axis is set as a light wavelength, and a vertical axis is set as a light intensity, and detected values from the spectrometer are displayed as a chart.

The storage section 5d stores the correction formula, the correction factor, and the graph used by the calculating section 34d in calculation. The storage section 5d stores the detected values from the Si photodetector 1d and the spectrometer 2d, and the like. The storage section 5d stores the control programs executed by the controller 3d.

(Flow of Processes of Controller 3d)

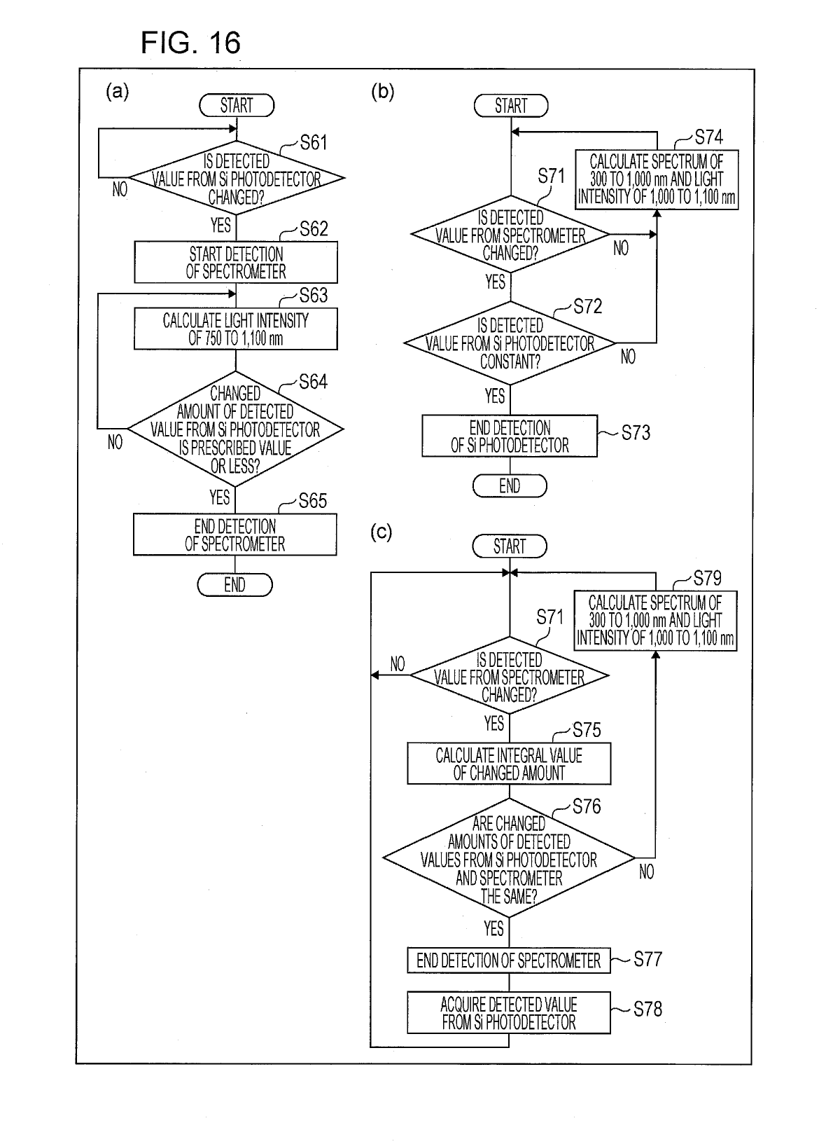

Subsequently, the flow of the data processes performed by the controller 3d is described by using FIG. 16(a). FIG. 16(a) is a flow chart illustrating a flow of the data processes performed by the controller 3d according to the embodiment.

The detected value acquiring section 31d receives the detected value from the Si photodetector 1d and sends the detected value to the sensor operation determining section 32d. The sensor operation determining section 32d determines whether the detected value from the Si photodetector 1d is changed (Step S61).

For example, in the determination, in a case where the change amount of the detected value from the Si photodetector 1d for each prescribed time is changed by two times or greater, or in a case where the detected value from the Si photodetector 1d at the time of detection start is changed by one time or greater, it is possible to determine that the detected value from the Si photodetector 1d is changed.

In a case where the sensor operation determining section 32d determines that the detected value from the Si photodetector 1d is changed (YES in Step S61), the sensor operation determining section 32d instructs the spectrometer 2d to start the detection (Step S62).

The detected value acquiring section 31d receives the detected values from the Si photodetector 1d and the spectrometer 2d and sends the detected values from the Si photodetector 1d and the spectrometer 2d to the calculating section 34d. The calculating section 34d calculates the light intensity in a wavelength of 750 to 1,100 nm from the detected values from the Si photodetector 1d and the spectrometer 2d (Step S63).

The sensor operation determining section 32d monitors the detected values from the Si photodetector 1d at a prescribed time interval and determines whether the change amount of the detected values from the Si photodetector 1d is within the prescribed value (Step S64).

In a case where the sensor operation determining section 32d determines that the change amount of the detected value from the Si photodetector 1d is within the prescribed value (YES in Step S64), the sensor operation determining section 32d instructs the spectrometer 2d to stop (end) the detection.

In a case where the sensor operation determining section 32d does not determine that the detected value from the Si photodetector 1d is changed (in a case of NO in Step S61), the detected value acquiring section 31d receives the detected value from the Si photodetector 1d and the process proceeds to Step S61.

In a case where the sensor operation determining section 32d determines that the change amount of the detected value from the Si photodetector 1d not within the prescribed value (NO in Step S64), the process proceeds to Step S63.

According to the configuration described above, in a case where it is determined that the detected value from the Si photodetector 1d is changed, the detection of the spectrometer 2d starts.

The change of the detected values from the Si photodetector 1d means a state in which the light in a specific wavelength performs penetration, reflection, absorption, diffusion, and emission with respect to the substance. That is, in the configuration of this embodiment, the detection of the spectrometer 2d starts when the light in a specific wavelength performs penetration, reflection, absorption, diffusion, and emission with respect to the substance. Since a spectrum can be measured in the spectrometer 2d, detailed analysis becomes possible. Accordingly, only when the specific analysis of the spectrometer 2d is required, the spectrometer 2d can be started. Accordingly, the power consumption of the light sensing system 10d can be reduced. The spectrometer 2d does not have to always perform detection. Therefore, the consumption generated according to the detection time can be suppressed. That is, the life span of the sensing system can be increased.

The detected value from the Si photodetector 1d and the detected value from the spectrometer 2d are analyzed together, so as to obtain detailed information.

The sensor operation determining section 32d may configure the detection condition (measuring time, a configured temperature, an applied voltage, and the like) of the spectrometer 2d from the detected value from the Si photodetector 1d, For example, according to the detected value from the Si photodetector 1d, the applied voltage may be adjusted such that the detection sensitivity of the spectrometer 2d becomes suitable sensitivity. Accordingly, it is not concerned that the spectrometer 2d breaks due to an input exceeding the detection limit.

The sensor operation determining section 32d determines the time duration for detecting the spectrometer 2d from the detected value from the Si photodetector 1d.

According to the configuration above, the detection condition described above influence on the detection sensitivity, the life span, and the power consumption of the spectrometer 2d and the like. Therefore, in view of detection sensitivity of the spectrometer 2d, the usage period of time of the spectrometer 2d, the period of time until the refreshment, the power consumption, the running cost, the reduction of the consumption, the detection condition of the suitable spectrometer 2d can be configured determining the detection condition described above from the detected value from the Si photodetector 1d in the light sensing system 10d.

With respect to the substance having an absorption peak specific to red by using the light sensing system 10d, an example of detecting the substance state change by using the penetration of light is illustrated below.

The light intensity of the red to infrared wavelength range that is detected by the Si photodetector 1d changes. The sensor operation determining section 32d starts the detection of the spectrometer 2d. The user can analyze whether the changes of the light intensity in a red to infrared wavelength range by checking the spectrum in 600 nm to 750 nm from the detected value from the spectrometer 2d are derived from a shift of the absorption peak or a change of an absorption amount.

For example, with respect to the target that detects the change of the substance state, if the substance having an absorption peak specific to read is metal fine particles, a change of a plasmon absorption peak can be known, and thus an optical constant change near the metal fine particles or form changes of the metal fine particles can be detected. Accordingly, a gas sensor or a liquid senor using a plasmon absorption peak is obtained. That is, the substance that generates the optical constant changes near the metal fine particles or the form changes of the metal fine particles is a target for detecting changes of the substance state and the detection purpose is to obtain information on the concentration of gas or liquid. The same applied to a substance having a specific light emission peak in red. At this point, if the emitted light is fluorescent, the detection of the spectrometer 2d is started, a spectrum at 300 nm to 750 nm is measured, and a relative ratio between intensity of an excitation wavelength and intensity of a fluorescent wavelength can be analyzed.

The spectrometer 2d may be substituted with a Si photodetector that can detect light in a red to infrared wavelength range (for example, 600 nm to 1,100 nm) by substituting the Si photodetector 1d to a spectrometer that can detect light in an ultraviolet to infrared region wavelength range (for example, in 300 nm to 1,100 nm).

The spectrometer 2d may be substituted with a spectrometer that can detect light in an ultraviolet to infrared wavelength range (for example, 300 nm to 1,100 nm) by substituting the Si photodetector 1d to a Si photodetector that can detect light in a red to infrared wavelength range (for example, 600 nm to 1,100 nm). In this case, according to the detected value from one detector, the sensor operation determining section 32d determines a detection operation of the other detector.

The light sensing system 10d may be mounted on a system that detects gas from a leaf of a plant, a system that detects components of a liquid fertilizer, a system that detects fluorescence of a leaf, and the like.

For example, in a case of detecting slight changes such as a growth state or a moisture amount of a leaf, all the detectors included in the light sensing system do not have to always perform detection. Therefore, it is effective to apply light sensing system 10d according to the embodiment.

In this embodiment, the light sensing system that detects light intensity is exemplified, but may be a sensing system, for example, for other continuous physical parameters (detection targets) such as vibration, sounds, radiation, and electron energy.

### MODIFICATION EXAMPLE 1

In this modification example, the first detector is the spectrometer 2d that can detect light in an ultraviolet to infrared region wavelength range (for example, 300 nm to 1,000 nm) and the second detector is the Si photodetector 1d that can detect light in a red to infrared wavelength range (for example, 600 nm to 1,100 nm). This modification example corresponds to a system that determines an operation of the first detector based on the second detection result.

The modification example of the flow of the data process performed by the controller 3d is described by using FIG. 16(b). FIG. 16(b) is a flow chart illustrating a flow of data processes performed by the controller 3d according to this modification example.

In this modification example, the Si photodetector 1d and the spectrometer 2d start operations together by the user starting an operation of the light sensing system 10d.

The sensor operation determining section 32d illustrated in FIG. 16(b) monitors the detected value from the spectrometer 2d at a prescribed time interval and determines whether the detected value from the spectrometer 2d is changed (Step S71).

In a case where the sensor operation determining section 32d determines that the detected value from the spectrometer 2d is changed (YES in Step S71), the sensor operation determining section 32d determines the change amount of the detected value from the Si photodetector 1d is within the prescribed value (Step S72).

In a case where it is determined that the change amount of the detected value from the Si photodetector 1d is within a prescribed value (YES in Step S72), the sensor operation determining section 32d instructs the Si photodetector 1d to stop (end) detection (Step S73).

In a case where the sensor operation determining section 32d determines that the detected value from the spectrometer 2d is not changed or in a case where the sensor operation determining section 32d determines that the change amount of the detected value from the Si photodetector 1d is not within the prescribed value (NO in Step S71 or S72), the calculating section 34d calculates a spectrum of 300 nm to 1,000 nm and the light intensity of 1,000 nm to 1,100 nm from the detection results of the Si photodetector 1d and the spectrometer 2d (Step S74). Thereafter, the process proceeds to Step S71.

According to the configuration, time for stopping the detection of the Si photodetector 1d can be provided. Therefore, the power consumption of the light sensing system 10d can be reduced.

### MODIFICATION EXAMPLE 2

Subsequently, a modification example of a flow of the data process performed by the controller 3d is described by using FIG. 16(c). FIG. 16(c) is a flow chart illustrating a flow of data processes performed by the controller 3d according to this modification example.

In this modification example, the Si photodetector 1d and the spectrometer 2d start operations together by the user starting an operation of the light sensing system 10d.

As illustrated in FIG. 16(c), the sensor operation determining section 32d monitors the detected value from the spectrometer 2d at a prescribed time interval and determines whether the detected value from the spectrometer 2d is changed (Step S71).

In a case where the sensor operation determining section 32d determines that the detected value from the spectrometer 2d is changed (YES in Step S71), the calculating section 34d calculates an integral value of a change amount (Step S75).

Subsequently, the sensor operation determining section 32d determines that the change amount of the detected value from the Si photodetector 1d is the same as the integral value of the change amount of the spectrometer 2d calculated by the calculating section 34d (Step S76). In a case where it is determined that the change amount of the detected value from the Si photodetector 1d is the same as the change amount and the integral value of the spectrometer 2d calculated by the calculating section 34d (YES in Step S76), the sensor operation determining section 32d instructs the spectrometer 2d to stop (end) the detection (Step S77).

Until the sensor operation determining section 32d monitors the detected value from the spectrometer 2d, the detected value acquiring section 31d acquires the detected value only from the Si photodetector 1d (Step S78). Thereafter, the process proceeds to Step S71.

In a case where the sensor operation determining section 32d determines that the detected value from the spectrometer 2d is not changed or in a case where the sensor operation determining section 32d determines that the change amount of the detected value from the Si photodetector 1d is not the same as the change amount and the integral value of the spectrometer 2d calculated by the calculating section 34d (NO in Step S71 or S72), the calculating section 34d calculates a spectrum of 300 nm to 1,000 nm and light intensity of 1,000 nm to 1,100 nm from the detection results of the Si photodetector 1d and the spectrometer 1d (Step S79). Thereafter, the process proceeds to Step S71.

The data process described in this modification example may have a configuration of receiving a data process ending instruction from the user and ending the data process.

According to the configuration, when it is determined that the Si photodetector 1d and the spectrometer 2d measure changes of the common detection targets, time at which the detection by the spectrometer 2d is stopped can be provided. Therefore, the power consumption of the light sensing system 10d can be reduced.

### Embodiment 5

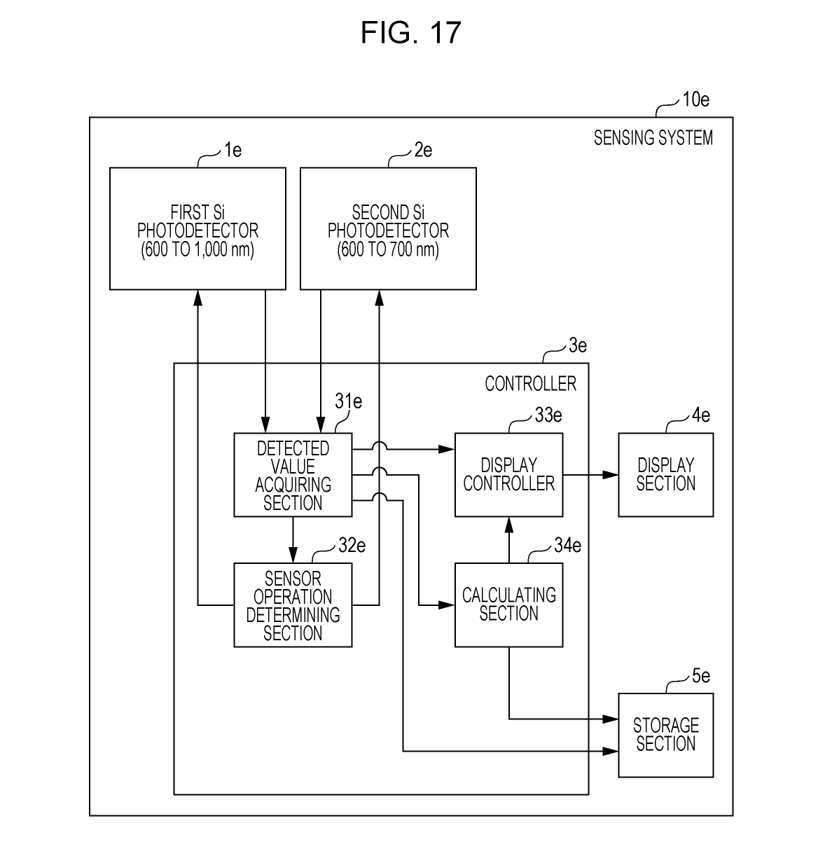

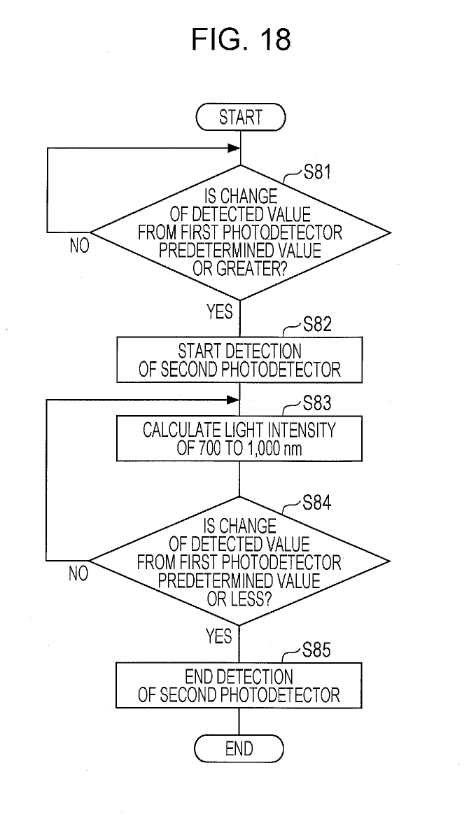

The embodiment according to the invention is described below based on FIGS. 17 and 18. For convenience of explanation, members having the same functions as members described in the embodiment are denoted by the same reference numerals, and the descriptions thereof are omitted.

The sensing system according to the embodiment is a light sensing system 10e (the sensing system 10) of which the detection purpose is to obtain information on existence or a state of a substance from the penetration, reflection, absorption, diffusion, light emission spectrum of light.

An example of the external appearance of the light sensing system 10e is the same as the light sensing system 10d described in Embodiment 4. Accordingly, the descriptions thereof are omitted.

(Main Configurations of Light Sensing System 10e)

Subsequently, main configurations of the light sensing system 10e according to the embodiment are described with reference to FIG. 17. FIG. 17 is a functional block diagram schematically illustrating a configuration of the light sensing system 10e. As illustrated in FIG. 17, the light sensing system 10e include a first Si photodetector le (first detector), a second Si photodetector 2e (second detector), a controller 3e, a display section 4e, and a storage section 5e.

(First Si Photodetector 1e)

The first Si photodetector 1e detect light in a red to infrared wavelength range (for example, 600 nm to 1,000 nm) as a detection target. The first Si photodetector 1e sends the detected value to the controller 3e.

(Second Si photodetector 2e)

The second Si photodetector 2e detects light in a specific wavelength range (for example, 600 nm to 700 nm) as a detection target. The second Si photodetector 2e sends the detected value to the controller 3e. For example, the second Si photodetector 2e is a configuration including a band pass filter that transmits only light in a specific wavelength range.

Here, the detection target of the first Si photodetector 1e and the detection target of the second Si photodetector 2e are detection targets included in the first concept which is the light intensity. The detection target common to the first Si photodetector 1e and the second Si photodetector 2e is light intensity at a wavelength of 600 nm to 700 nm included in the subordinate concept of the first concept.

The first Si photodetector 1e is set as a Si photodetector with a band pass filter and may be the second Si photodetector 2e.

(Controller 3e)

The controller 3e includes a detected value acquiring section 31e, a sensor operation determining section 32e, a display controller 33e, and a calculating section 34e.

The detected value acquiring section 31e receives the detected values from the first Si photodetector 1e and the second Si photodetector 2e and sends the detected values to the sensor operation determining section 32e, the display controller 33e, the calculating section 34e, and the storage section 5e.

The sensor operation determining section 32e determines the detected values from the first Si photodetector 1e and/or the second Si photodetector 2e and controls the detection operations of the first Si photodetector 1e or the second Si photodetector 2e.

The control of the detection operation is described in “flow of processes of controller 3e” is specifically described.

The display controller 33e receives the detected value from the first Si photodetector 1e or the second Si photodetector 2e from the detected value acquiring section 31e or the light intensity calculated by the calculated section 34e and instructs the display section 4e to display the detected value and the light intensity in respective wavelength regions on the display section 4e.

If the detected value is disclosed to the user, the user can check whether the light sensing system 10e normally functions. The user can manipulate the control condition such as sensitivity of the respective photodetectors based on the display data by adjusting a circuit constant relating to the first Si photodetector 1e and/or the second Si photodetector 2e.

The display controller 33e may perform instruction such that the light intensity calculated from the detected values from the first Si photodetector 1e and the second Si photodetector 2e to be displayed. A growth state and a moisture amount of a leaf and the like determined from the detected values from the first Si photodetector 1e and the second Si photodetector 2e may be displayed.

The calculating section 34e calculates the detected values of the light intensity in a wavelength of 700 to 1,000 nm from the detected values from the first Si photodetector 1e and the second Si photodetector 2e. The calculated value is sent to the display controller 33e and the storage section 5e.

The calculating section 34e may exclude a detected values (600 to 700 nm) of the detection target common to both of the first Si photodetector 1e and the second Si photodetector 2e from the detected values from the first Si photodetector 1e and set a detected value (detected value of 700 to 1,000 nm) of the detection target, which is not common to the second Si photodetector 2e, of the first Si photodetector 1e as the detected value from the first Si photodetector 1e.

A calculation method described in the calculation of the detected value of ethanol from the calculating section 34a described in Embodiment 1 can be applied to the calculation of the detected values of the light intensity in a wavelength of 700 to 1,000 nm. The detected value of the light intensity and the light intensity in a wavelength of 700 to 1,000 nm may be calculated, for example, by substituting the detected value from the hygrometer 1a in the calculation of the detected value of ethanol in Embodiment 1 to the detected value from the second Si photodetector 2e and substituting the detected value from the semiconductor gas sensor 2a in Embodiment 1 to the detected value from the first Si photodetector 1e.

The storage section 5e stores the correction formula, the correction factor, and the graph used by the calculating section 34e in calculation. The storage section 5e stores the detected values from the first Si photodetector 1e and the second Si photodetector 2e, and the like. The storage section 5e stores the control program executed by the controller 3e.

(Flow of Processes of Controller 3e)

Subsequently, the flow of the data processes performed by the controller 3e is described by using FIG. 18. FIG. 18 is a flow chart illustrating a flow of data processes performed by the controller 3e according to the embodiment.

The detected value acquiring section 31e receives the detected value from the first Si photodetector 1e and sends the detected value to the sensor operation determining section 32e. The sensor operation determining section 32e determines that the detected value from the first Si photodetector 1e is changed (Step S81).

For example, in the determination, in a case where the change amount of the detected value from the first Si photodetector 1e for each prescribed time is changed two times or greater or in a case where the detected value from the first Si photodetector 1e at the time of detection start is changed by 10% or greater, it is possible to determine that the detected value from the first Si photodetector 1e is changed.

In a case where the sensor operation determining section 32e determines that the detected value from the first Si photodetector 1e is changed (YES in Step S81), the sensor operation determining section 32e instructs the second Si photodetector 2e to start the detection (Step S82).

If the detected value acquiring section 31e receives the detected values from the first Si photodetector 1e and the second Si photodetector 2e, the detected values from the first Si photodetector 1e and the second Si photodetector 2e are sent to the calculating section 34e. The calculating section 34e calculates the light intensity in a wavelength of 700 to 1,000 nm from the detected values from the first Si photodetector 1e and the second Si photodetector 2e (Step S83).

The sensor operation determining section 32e monitors the detected value from the first Si photodetector 1e at a prescribed time interval and determines whether the change amount of the detected value from the first Si photodetector 1e is within the prescribed value (Step S84).

In a case where the sensor operation determining section 32e determines that the change amount of the detected value from the first Si photodetector 1e is within the prescribed value (YES in Step S84), the sensor operation determining section 32e instructs the second Si photodetector 2e to stop (end) the detection.

In a case where the sensor operation determining section 32e does not determine that the detected value from the first Si photodetector 1e is changed (in a case of NO in Step S81), the detected value acquiring section 31e receives the detected value from the first Si photodetector 1e and the process proceeds to Step S81.

In a case where the sensor operation determining section 32e determines that the change amount of the detected value from the first Si photodetector 1e is within the prescribed value (NO in Step S84), the process proceeds to Step S83.

In the configuration above, in a case where it is determined that the detected value from the first Si photodetector 1e is changed, the detection of the second Si photodetector 2e starts.

The change of the detected value from the first Si photodetector 1e occurs due to penetration, reflection, absorption, diffusion, and emission of light in a specific wavelength in the detection target of the first Si photodetector 1e with respect to the substance. That is, the configuration of this embodiment is a configuration in which the detection of the second Si photodetector 2e starts, when the light in a specific wavelength in the detection target of the first Si photodetector 1e becomes in a state of penetration (a state of reflection, absorption, diffusion, or emission is possible).

If the detection of the second Si photodetector 2e starts, the second Si photodetector 2e detects only light in a limited wavelength range (600 nm to 700 nm) among light being the detection targets (600 nm to 1,000 nm) of the first Si photodetector 1e. Therefore, it is possible to determine that whether the change of the detected values from the first Si photodetector 1e is the detection target of the second Si photodetector 2e or a change of another detection target. That is, the light sensing system 10e can independently (selectively) detect the light intensity in 600 nm to 700 nm and in 700 nm to 1,000 nm.

The light sensing system 10e can approximately determine a phenomenon that occurs in a substance of an observation target with a simple configuration including two Si photodetectors and a band pass filter. A detection target which is common to both of the detection target of the first Si photodetector 1e and the detection target of the second Si photodetector 2e exists (the detection target of the second Si photodetector 2e is included in the detection target of the first Si photodetector 1e). Therefore, it is not required to always operate the second Si photodetector 2e and thus it is possible to increase the life span and reduce the power consumption of the light sensing system 10e.

For example, the sensor operation determining section 32e starts the detection operation of the second Si photodetector 2e according to the detected value from the first Si photodetector 1e. Thereafter, in a case where the sensor operation determining section 32e determines that the change of the detected value from the first Si photodetector 1e changes according to the change of the detected value from the second Si photodetector 2e (there is no change in the detection targets other than the detection target of the second Si photodetector 2e), the sensor operation determining section 32e may instruct the first Si photodetector 1e to stop the detection.

According to the configuration, time at which the detection of the first Si photodetector 1e is stopped can be provided. Therefore, the power consumption of the light sensing system 10e can be reduced.

The light sensing system 10e can be used for the detection of a change in a state of a substance of which penetration, reflection, and absorption peaks with respect to light at a wavelength of 600 nm to 700 nm are changed by a specific phenomenon. That is, it the detected value from the first Si photodetector 1e changes (the light intensity in a red to infrared wavelength range changes), the sensor operation determining section 32e starts the detection by the second Si photodetector 2e. Therefore, it is possible to analyze whether the change of the detected value from the first photodetector 1e is a change of the light intensity in 600 nm to 700 nm. Accordingly, the light sensing system 10e can be used for obtaining information on a change in a state of a substance of which penetration, reflection, and an absorption peak with respect to light in a wavelength of 600 nm to 700 nm are changed due to a specific phenomenon.

Here, obtaining the information on the change of the state of the substance of which the penetration, reflection, and an absorption peak with respect to the light in a wavelength of 600 nm to 700 nm due to specific phenomenon by using the light sensing system 10e is specifically described.

For example, the specific phenomenon is decomposition of chlorophyll which is a dye of a chloroplast of a plant and the detection purpose thereof is to obtain information on the change of the state of the leaf.

The first Si photodetector 1e always detects light in a red to infrared wavelength range (600 nm to 1,000 nm). If the detected value from the first Si photodetector 1e increases or decreases by a prescribed value or greater, the sensor operation determining section 32e starts the detection of the second Si photodetector 2e. The second Si photodetector 2e detects the light intensity of a limited wavelength range (600 nm to 700 nm) among light being the detection targets (600 nm to 1,000 nm) of the first Si photodetector 1e.

Accordingly, from the detected value from the second Si photodetector 2e, the user can determine whether the change of the detected value from the first Si photodetector 1e is the change of the light intensity in a wavelength of 600 nm to 700 nm which have one absorption peak of chlorophyll or a change in other light intensity in a wavelength of 600 nm to 700 nm (for example, the change of fluorescence in a wavelength region of the detection target of the first Si photodetector 1e and the decrease of absorption in an infrared range caused by the deficiency of the moisture amount in the leaf). For example, the calculating section 34e compares the intensity change of 600 nm to 700 nm and the intensity change of 600 nm to 1,000 nm so as to perform this determination based on the corresponding comparison. This determination may be performed by the calculating section 34e, but the invention is not limited thereto.

It is possible to detect temporal changes of the detected value from the first Si photodetector 1e and the detected value from the second Si photodetector 2e by simultaneously detecting the first Si photodetector 1e and the second Si photodetector 2e.

In this embodiment, the second Si photodetector 2e is set as a Si photodetector with a band pass filter but the second Si photodetector 2e may be another type of detector having higher sensitivity than the first Si photodetector 1e. In this case, a detector having high sensitivity generally has a low detection limit, and thus if the detection operation is always performed, it is concerned that the detector breaks due to an input exceeding the detection limit. If the sensor operation determining section 32e starts the detection of the second detector from the detected value from the first Si photodetector 1e, the concern can be reduced.

The sensor operation determining section 32e may have a configuration of determining the detection condition of the second Si photodetector 2e from the detected value from the first Si photodetector 1e. In this case, the corresponding detection condition may influence on the detection sensitivity on the detection target of the second Si photodetector 2e, the life span of the second Si photodetector 2e, the power consumption of the second Si photodetector 2e, and the like. In view of the detection sensitivity with respect to the detection target of the second Si photodetector 2e, the life span of the second Si photodetector 2e, the period of time until the refreshment of the second Si photodetector 2e, and the power consumption of the second Si photodetector 2e, the sensor operation determining section 32e may configure the suitable detection condition of the second Si photodetector 2e.

The light sensing system 10e may be mounted on a system that detects gas from a leaf of a plant, a system that detects components of a liquid fertilizer, a system that detects fluorescence of a leaf, and the like.

For example, in a case of detecting slight changes such as a growth state or a moisture amount of a leaf, all the detectors included in the light sensing system do not have to always perform detection. Therefore, it is effective to apply the light sensing system 10e according to the embodiment.

In this embodiment, the light sensing system that detects light intensity is exemplified, but may be a sensing system, for example, for other continuous physical parameters (detection targets) such as vibration, sounds, radiation, and electron energy.

[Realization Example of Software]

Control blocks 3a to 3e of the sensing systems 10a to 10e may be realized by a logical circuit (hardware) formed with an integrated circuit (IC chip), and the like or may be realized by software by using a central processing unit (CPU).

In the case of the latter, the sensing systems 10a to 10e each include a CPU that executes instructions of programs that are software realizing respective functions, a read only memory (ROM) or a storage device (also referred to as a “recording medium”) in which the programs and respective types of data are recorded readable by computer (or CPU), a random access memory (RAM) in which the programs are developed, and the like. Also, if the computer (or CPU) executes reading the programs from the recording medium, the purpose of the invention is achieved. As the recording medium, a “medium that is not temporary”, for example, a tape, a disc, a card, a semiconductor memory, and a programmable logical circuit can be used. The programs may be supplied to the computer via an arbitrary transmission medium (communication networks, broadcast waves, and the like) that can transmit the programs. The invention may be realized in a form of data signals embedded in carrier waves in which the programs are realized by electronic transmission.

[Additional Notes]

The invention can be presented as follows.

A sensing system includes: a first detector that detects a first detection target; a second detector that detects a second detection target; and a controller that controls start or stop of detection operations of the first detector and the second detector, the first detection target and the second detection target are detection targets included in a first concept and include at least one type of detection target which is common to both of the first detection target and the second detection target and is included in a subordinate concept of the first concept, and, according to the detected value from any one detector of the first detector and the second detector, the controller controls start, stop, or a detection condition of the detection operation of the other detector.

A sensing system in which, in a case where the first detection target only includes a detection target common to the second detection target and the second detection target includes a detection target other than the common detection target, the controller controls start or stop of a detection operation by the second detector according to the detected value of the common detection target included in the first detection target detected by the first detector.

A sensing system in which, in a case where the first detection target includes a detection target other than the detection target common to the second detection target and the second detection target includes only the common detection target, the controller controls starting or stop of the detection operation by the second detector according to the detected value of the first detection target detected by the first detector.

A sensing system, in which, in a case where all the detection targets included in the first detection target and the second detection target are the same, the controller performs control such that detected temperatures of the first detector and the second detector are different from each other.

### CONCLUSION

According to a sensing system (the sensing system 10, the alcohol detecting system 10a, the air quality monitoring system 10b, the gas sensing system 10c, the light sensing system 10d, or the light sensing system 10e) of a first aspect of the invention includes a first detector (the first detector 1, the hygrometer 1a, the semiconductor gas sensor 1b, the first semiconductor gas sensor 1c, the Si photodetector 1d, or the first Si photodetector 1e) that detects a first detection target; a second detector (the second detector 2, the semiconductor gas sensor 2a, the light absorbance-type gas sensor 2b, the second semiconductor gas sensor 2c, the spectrometer 2d, or the second Si photodetector 2e) that detects a second detection target; and a controller (the controller 3, the controllers 3a to 3e, or the sensor operation determining sections 32a to 32e) that controls start or stop of detection operations of the first detector and the second detector, in which the first detection target and the second detection target are detection targets included in a first concept and include at least one type of detection target which is common to both of the first detection target and the second detection target and is included in a subordinate concept of the first concept, and, according to the detected value from any one detector of the first detector and the second detector, the controller controls start, stop, or a detection condition of the detection operation of the other detector.

In the configuration, according to the detected value from any one detector of the first detector and the second detector, the controller controls starting, stopping, or detection conditions of a detection operation of the other detector. For example, in the configuration of controlling start or stop of the detection operation of the other detector according to the detected value from one detector, all the detector do not have to always perform detection. Accordingly, it is possible to suppress the consumption generated according to the detection time of the respective detectors. That is, the sensing system of the sensing system can be increased.

The first detection target and the second detection target are detection targets included in a first concept and include at least one type of detection target which is common to both of the first detection target and the second detection target and is included in a subordinate concept of the first concept. Each of the detectors can obtain a detection result of each other according to the at least one type of detection target which is common to both of the first detection target and the second detection target and which is included in a subordinate concept of the first concept.

In the configuration in which, according to the detected value from one detector, the detection condition of the detection operation of the other detector is controlled, for example, the detector of which the detection condition is controlled is a semiconductor film-type gas sensor. According to the control of the detection condition, in a case where the detected temperature is set to be low, a reaction film of the semiconductor film-type gas sensor hardly degraded, compared with the configuration in which the detected temperature is set to as a high temperature. Therefore, the life span of the sensing system can be increased.

According to a sensing system of a second aspect of the invention, in the first aspect, a calculating section (the calculating sections 34a to 34e) is further included, and the calculating section may exclude a detected value of the detection target which is common to the both from the detected values of the detection targets of the one detector and may set a detected value of the detection target, which is not common to the other detected value, of the one detector, as the detected value of the one detector.

According to the configuration, the calculating section calculates the detected value of the detection target, which is not common to the other detector, of the one detector.

Accordingly, in the detection target of the one detector, the detected value of the detection target, which is not common to the other detector, of the one detector can be selectively calculated.

According to a sensing system (the alcohol detecting system 10a) of a third aspect of the invention, in the first aspect,

according to a detected value of any one detector of the first detector and the second detector, the controller (the controller 3a or the sensor operation determining section 32a) controls a detection condition of a detection operation of the other detector, the first or second detector is a semiconductor film-type gas sensor (the semiconductor gas sensor 1b), and the controller (the controller 3a or the sensor operation determining section 32a) may control a temperature of a reaction film of the semiconductor film-type gas sensor and may perform control such that the semiconductor film-type gas sensor does not detect at least one type of detection target which is common to the both and is included in a subordinate concept of the first concept.

According to the configuration, the controller, by controlling the temperature of the reaction film of the semiconductor film-type gas sensor, performs control such that the at least one type of detection target which is common to the both and is included in the subordinate concept of the first concept is not detected.

Accordingly, one detector can detect a detection target except for at least one type of detection target which is common to the both and is included in the subordinate concept of the first concept.

According to the sensing system (the alcohol detecting system 10a, the air quality monitoring system 10b, the light sensing system 10d) of a fourth aspect of the invention, in the first to third aspects, a detection principle of the first detector (the hygrometer 1a, the semiconductor gas sensor 1b, or the Si photodetector 1d) and a detection principle of the second detector (semiconductor gas sensor 2a, the light absorbance-type gas sensor 2b, and the spectrometer 2d) are different from each other.

According to the configuration, start or stop (end) of detection of a detector having performances of hard refreshment, high running cost, and being wasted, high power consumption can be controlled according to the detected value from the other detector.

Accordingly, the detector of which the start or stop (end) is controlled is not required to always perform detection.

Accordingly, according to the configuration, it is possible to realize a sensing system having low power consumption, low cost, and a long life span.

According to a sensing system (the gas sensing system 10c and the light sensing system 10e) of a fifth aspect of the invention, in the first to third aspects, a detection principle of the first detector (the first semiconductor gas sensor 1c or the first Si photodetector 1e) and a detection principle of the second detector (the second semiconductor gas sensor 2c or the second Si photodetector 2e) are the same and detection conditions of the first detector and the second detector are different from each other.

According to the configuration, the two detectors have the same detection principle. Therefore, since the control method of each of the detectors is common, it is possible to reduce process amounts of control processes of the two detectors.

For example, in a case where two semiconductor film-type gas sensors having the same detection principles are respectively have different types of reaction films, start or end of the detection of the detector including a reaction film that is hardly refreshed can be controlled according to the detected value from the other detector. Accordingly, it is possible to start the detection of the detector including a reaction film that is hardly refreshed, only when necessary.

Accordingly, if the detector is not operated, the detection target does not react with the reaction film, and thus it is possible to cause the saturation of the reaction between the detection target and the reaction film of the detector to be slow.

Accordingly, it is possible to suppress the number of refreshment of the reaction film performed at the time of the corresponding saturation state. Since the temperature of the reaction film is generally increased in order to refresh the reaction film, it is possible to achieve a long life span of the detector of the sensing system by suppressing the number of refreshment and preventing the degradation of the reaction film.

The sensing system according to the respective aspects of the invention may be realized by a computer. In this case, a control program of the sensing system that realizes the sensing system with the computer by causing the computer to be operated as respective sections included in the sensing system and a computer readable recording medium in which the control program is stored are included in the invention.

The invention is not limited to the respective embodiments, and various changes can be made within the scope recited in the claims, and embodiments that can be obtained by appropriately combining technical means respectively disclosed in other embodiments are also included in the technical scope of the invention. It is possible to form new technical features by combining technical means described in the respective embodiments.

### INDUSTRIAL APPLICABILITY

The invention can be used in a sensing system.

### REFERENCE SIGNS LIST

1 first detector

1a hygrometer (first detector)

1b semiconductor gas sensor (first detector)

1c first semiconductor gas sensor (first detector)

1d Si photodetector (first detector)

1e first Si photodetector (first detector)

2 second detector

2a semiconductor gas sensor (second detector)

2b gas sensor (second detector)

2b light absorbance-type gas sensor (second detector)

2c second semiconductor gas sensor (second detector)

2d spectrometer (second detector)

2e second Si photodetector (second detector)

3, 3a to 3e controller

10 sensing system

10a alcohol detecting system (sensing system)

10b air quality monitoring system (sensing system)

10c gas sensing system (sensing system)

10d light sensing system (sensing system)

10e light sensing system (sensing system)

32a to 32e sensor operation determining section (controller)

34a to 34e calculating section

1. A sensing system comprising:
a first detector that detects a first detection target;
a second detector that detects a second detection target; and
a controller that controls start or stop of detection operations of the first detector and the second detector,
wherein the first detection target and the second detection target are detection targets included in a first concept and include at least one type of detection target which is common to both of the first detection target and the second detection target and is included in a subordinate concept of the first concept, and
wherein, according to a detected value from any one detector of the first detector and the second detector, the controller controls start, stop, or a detection condition of the detection operation of the other detector.

2. The sensing system according to claim 1,
wherein the controller includes a calculating section, and
wherein the calculating section excludes a detected value of the detection target which is common to the both from detected values of the detection targets of the one detector and sets a detected value of the detection target, which is not common to the other detector, of the one detector, as the detected value of the one detector.

3. The sensing system according to claim 1,
wherein, according to a detected value of any one detector of the first detector and the second detector, the controller controls a detection condition of a detection operation of the other detector,
wherein the first detector or the second detector is a semiconductor film-type gas sensor,
wherein the controller controls start of the detection of the first detector or the second detector, and
wherein the controller, by controlling a temperature of a reaction film of the semiconductor film-type gas sensor, performs control so that the semiconductor film-type gas sensor does not detect at least one type of detection target which is common to the both and is included in the subordinate concept of the first concept.

4. The sensing system according to claim 1,
wherein a detection principle of the first detector and a detection principle of the second detector are different from each other.

5. The sensing system according to claim 1,
wherein a detection principle of the first detector and a detection principle of the second detector are the same and detection conditions of the first detector and the second detector are different from each other.
